(19) United States
(12) Patent Application Publication
(10) Pub. No.: US 2011/0195973 A1
(43) Pub. Date: Aug. 11, 2011

(54) CYCLIC SUBSTITUTED PYRAZINOYLGUANIDINE SODIUM CHANNEL BLOCKERS POSSESSING BETA AGONIST ACTIVITY

(75) Inventor: Michael Ross Johnson, Chapel Hill, NC (US)

(73) Assignee: PARION SCIENCES, Inc., Durham, NC (US)

(21) Appl. No.: 12/304,042

(22) PCT Filed: Jun. 11, 2007
(86) PCT No.: PCT/US07/70862

§ 371 Date: Jan. 26, 2009

Related U.S. Application Data
(60) Provisional application No. 60/812,091, filed on Jun. 9, 2006.

(51) Int. Cl. A61K 31/497; C07D 241/28; A61K 31/4965; C07D 403/12; A61P 11/00; A61P 27/02; A61P 17/00

(52) U.S. Cl. 514/252.11; 544/407; 514/255.06; 514/255.05; 544/357

(57) ABSTRACT

The present invention relates to sodium channel blockers. The present Invention also includes a variety of methods of treatment using these inventive sodium channel blockers.

### CONTINUING APPLICATION DATA

This application claims priority to U.S. provisional application Ser. No, 60/812,091, filed on Jun. 9, 2006, and incorporated herein by reference,

### BACKGROUND OF THE INVENTION

1. Field of the Invention

The present invention relates to sodium channel blockers possessing beta-adrenergic receptor agonist activity. The present invention also includes a variety of methods of treatment using these inventive sodium channel blockers/beta-adrenergic receptor agonists.

2. Description of the Background

The mucosal surfaces at the interface between the environment and the body have evolved a number of “innate defenses”, i.e., protective mechanisms. A principal form of such an innate defense is to cleanse these surfaces with liquid. Typically, the quantity of the liquid layer on a mucosal surface reflects the balance between epithelial liquid secretion, often reflecting anion (Cl⁻ and/or HCO₃⁻) secretion coupled with water (and a cation counter-ion), and epithelial liquid absorption, often reflecting Na⁺ absorption, coupled with water and counter anion (Cl⁻ and/or HCO₃⁻). Many diseases of mucosal surfaces are caused by too little protective liquid on those mucosal surfaces created by an imbalance between secretion (too little) and absorption (relatively too much), The defective salt transport processes that characterize these mucosal dysfunctions reside in the epithelial layer of the mucosal surface.

One approach to replenish the protective liquid layer on mucosal surfaces is to “re-balance” the system by blocking Na⁺ channel and liquid absorption and simultaneously activating beta-adrenergic receptors thereby causing liquid secretion. The epithelial protein that mediates the rate-limiting step of Na⁺ and liquid absorption is the epithelial Na⁺ channel (ENaC), ENaC and beta-adrenergic receptors are positioned on the apical surface of the epithelium. i.e. the mueosal surface-extermal environment interface. Therefore, to inhibit ENaC mediated Na⁺ and liquid absorption, an ENaC blocker of the amiloride class (which blocks from the extracellular domain of ENaC) must be delivered to the mucosal surface and, importantly, be maintained at this site, to achieve therapeutic utility. The present invention describes diseases characterized by too little liquid on mucosal surfaces and “topical” sodium channel blockers containing beta-adrenergic receptor agonist activity designed to exhibit the increased potency, reduced rnucosal abosorption, and slow dissociation (“unbinding” or detachment) from ENaC and the beta-adrenergic receptor required for therapy of these diseases.

Chronic bronchitis (CB), including the most common lethal genetic form of chronic bronchitis, cystic fibrosis (CF), are diseases that reflect the body's failure to clear mucus normally from the lungs, which ultimately produces chronic airways infection. In the normal lung, the primary defense against chronic intrapulmonary airways infection (chronic bronchitis) is mediated by the continuous clearance of mucus from bronchial airway surfaces. This function in health subjects effectively removes from the lung potentially noxious toxins and pathogens. Recent data indicate that the initiating problem, i.e., the “basic defect,” in both CB and CF is the failure to clear mucus from airway surfaces. The failure to clear mucus reflects an imbalance between the amount of liquid and mucin on airway surfaces. This “airway surface liquid” (ASL) is primarily composed of salt and water in proportions similar to plasma (i.e., isotonic). Mucin macromolecules are organized into a well defined “mucus layer” which normally traps inhaled bacteria and are transported out of the lung via the actions of cilia which beat in a watery, low viscosity solution termed the “periciliary liquid” (PCL). In the disease state, there is an imbalance in the quantities of mucus and ASL on airway surfaces. This imbalance results in a relative reduction in ASL which leads to mucus concentration, a reduction in the lubricant activity of the PCL, and a failure to clear mucus via ciliary activity to the mouth. The reduction in mechanical clearance of mucus from the lung leads to chronic bacterial colonization of mucus adherent to airway surfaces. It is the chronic retention of bacteria, the failure of local antimicrobial substances to kill mucus-entrapped bacteria on a chronic basis, and the consequent chronic inflammatory responses of the body to this type of surface infection, that lead to the syndromes of CB and CF.

The current afflicted population in the U.S. is 12,000,000 patients with the acquired (primarily from cigarette smoke exposure) form of chronic bronchitis and approximately 30,000 patients with the genetic form, cystic fibrosis. Approximately equal numbers of both populations are present in Europe. In Asia, there is little CF but the incidence of CB is high and, like the rest of the world, is increasing,

There is currently a large, unmet medical need for products that specifically treat CB and CF at the level of the basic defect that cause these diseases. The current therapies for chronic bronchitis and cystic fibrosis focus on treating the symptoms and/or the late effects of these diseases. Thus, for chronic bronchitis, inhaled β-agonists, steroids, anti-cholinergic agents, and oral theophyllines and phosphodiesterase inhibitors are all in current use. However, none of these drugs alone effectively treat the fundamental problem of the failure to clear mucus from the lung. Similarly, in cystic fibrosis, the same spectrum of pharmacologic agents are used. These strategies have been complemented by more recent strategies designed to clear the CF lung of the DNA (“Pulmozyme”; Genentech) that has been deposited in the lung by neutrophils that have futilely attempted to kill the bacteria that grow in adherent mucus masses and through the use of inhaled antibiotics (e.g, “TOBI”) designed to augment the lungs' own killing mechanisms to rid the adherent mucus plaques of bacteria. A general principle of the body is that if the initiating lesion is not treated, in this case mucus retention/obstruction, bacterial infections become chronic and increasingly refractory to antimicrobial therapy. Thus, a major unmet therapeutic need for both CB and CF lung diseases is an effective means of re-hydrating airway mucus (i.e., restoring/expanding the volume of the ASL) and promoting its clearance, with bacteria, from the lung.

R. C. Boucher, in U.S. Pat. No. 6,264,975, describes the use of pyrazinoylguanidine sodium channel blockers for hydrating mucosal surfaces. These compounds, typified by the well-known diuretics amiloride, benzamil, and phenamil, are effective. However, these compounds suffer from the significant disadvantage that they are (1) relatively impotent, which is important because the mass of drug that can be inhaled by the lung is limited; (2) rapidly absorbed, which limits the half-life of the drug on the mucosal surface; and (3) are freely dissociable from ENaC. The sum of these disadvantages embodied in these well known diurectics produces compounds with insufficient potency and/or effective half-life on mucosal surfaces to have therapeutic benefit for hydrating mucosai surfaces.

Clearly, what is needed are drugs that are more effective at restoring the clearance of mucus from the lungs of patients with CB/CF. The value of these new therapies will be reflected in improvements in the quality and duration of life for both the CF and the CB populations.

Other mucosal surfaces in and on the body exhibit subtle differences in the normal physiology of the protective surface liquids on their surfaces but the pathophysiology of disease reflects a common theme, i.e., too little protective surface liquid. For example, in xerostomia (dry mouth) the oral cavity is depleted of liquid due to a failure of the parotid sublingual and submandibular glands to secrete liquid despite continued Na⁺ (ENaC) transport mediated liquid absorption from the oral cavity. Similarly, keratoconjunctivitis sira (dry eye) is caused by failure of lacrimal glands to secrete liquid in the face of continued Na⁺ dependent liquid absorption on conjunctional surfaces. In rhinosinusitis, there is an imbalance, as in CB, between mucin secretion and relative ASL depletion. Finally, in the gastrointestinal tract, failure to secrete Cl— (and liquid) in the proximal small intestine, combined with increased Na⁺ (and liquid) absorption in the terminal ileum leads to the distal intestinal obstruction syndrome (DIOS). In older patients, excessive Na⁺ (and volume) absorption in the descending colon produces chronic constipation and diverticulitis.

### SUMMARY OF THE INVENTION

It is an object of the present invention to provide compounds that have both sodium channel blocking activity and beta-adrenergic receptor agonist activity in the same molecule.

It is an object of the present invention to provide compounds that are more potent and/or absorbed less rapidly from mucosal surfaces, and/or are less reversible as compared to known compounds.

It is another aspect of the present invention to provide compounds that are more potent and/or absorbed less rapidly and/or exhibit less reversibility, as compared to compounds such as amilorde, benzamil, and phenamil. Therefore, the compounds will give a prolonged phamacodynamic half-life on mucosal surfaces as compared to known compounds.

It is another object of the present invention to provide compounds which are (1) absorbed less rapidiy from mucosal surfaces, especially airway surfaces, as compared to known compounds and; (2) when absorbed from musosal surfaces after administration to the mucosal surfaces, are converted in vivo into metabolic derivitives thereof which have reduced efficacy in blocking sodium channels and beta-adrenergic receptor agonist activity as compared to the administered parent compound.

It is another object of the present invention to provide compounds that are more potent and/or absorbed less rapidly and/or exhibit less reversibility, as compared to compounds such as amiloride, benzamil, and phenamil. Therefore, such compounds will give a prolonged pharmacodynamic half-life on mucosal surfaces as compared to previous compounds.

It is another object of the present invention to provide methods of treatment that take advantage of the pharmacological properties of the compounds described above.

In particular, it is an object of the present invention to provide methods of treatment which rely on rehydration of mucosal surfaces.

Any of the compounds described herein can be a pharmaceutically acceptable salt thereof, and wherein the above compounds are inclusive of all racemates, enantiomers, diastereomers, tautomers, polymorphs and pseudopolymorphs thereof. Polyrnorphs are different physical forms—different crystal forms that have differing melting ranges, show differing differential scanning calorimetry (DSC) tracings and exhibit different X-Ray powder diffraction (XRPD) spectra. Pseudopolymorphs are different solvated physical forms—different crystal forms that have differing melting ranges as solvates, show differing differential scanning calorimetry (DSC) tracings as solvates and exhibit different X-Ray powder diffraction (XRPD) spectra as solvates.

The present invention also provides pharmaceutical compositions which contain a compound described above.

The present invention also provides a method of promoting hydration of mucosal surfaces, comprising:

administering an effective amount of a compound represented by formula (I) to a mucosal surface of a subject.

The present invention also provides a method of restoring mucosal defense, comprising:

topically administering an effective amount of compound represented by formula (I) to a mucosal surface of a subject in need thereof.

The present invention also provides a method of blocking ENaC and exe ng beta-adrenergic receptor agonism comprising:

contacting sodium channels and at the same time activating beta-adrenergic receptors (beta aonists) with an effective amount of a compound represented by formula (I).

The objects of the resent invention may be accomplished with a class of pyrazinoylguanidine compounds representing a compound represented by formula (I):

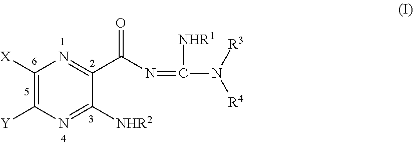

wherein

X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;

Y is hydrogen, hydroxyl, mercapto, lower alkoxy, lower alkyl-thio, halogen, lower alkyl, unsubstituted or substituted mononuclear aryl, or —N(R²)₂;

R¹ is hydrogen or lower alkyl;

each R² is, independently, —R⁷, —(CH₂)_{m—OR}⁸, —(CH₂)ₘ—NR⁷R¹⁰, —(CH₂)ₙ(CHOR⁸)(CHOR⁸)ₘ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ—Z_{g}—R⁷, —(CH₂)ₘ—NR¹⁰—CH₂(CHOR⁸)(CHOR⁸)ₙ—CH₂OR⁸, —(CH₂)ₙ—CO₂R⁷, or

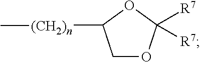

R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A):

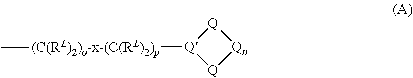

wherein each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

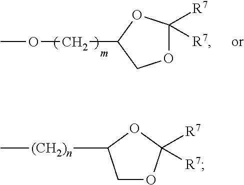

each o is, independently, an integer from 0 to 10;

each p is an integer from 0 to 10;

with the proviso that the sum of o and p in each contiguous chain is from 1 to 10;

each x is, independently, O, NR¹⁰, C(═O), CHOH, C(═N—R¹⁰), CHNR⁷R¹⁰, or represents a single bond;

wherein each R⁵ is, independently,

Link —(CH₂)ₙ—CR¹¹R¹¹—CAP, Link —(CH₂)ₙ(CHOR⁸)(CHOR⁸)ₙ—CR¹¹R¹¹—CAP, Link —(CH₂CH₂O)ₘ—CH₂—CR¹¹R¹¹—CAP, Link —(CH₂CH₂O)ₘ—CH₂CH₂—CR¹¹R¹¹—CAP, Link —(CH₂)ₙ—(Z)_{g}—CR¹¹R¹¹—CAP, Link —(CH₂)ₙ(Z)_{g}—(CH₂)ₘ—CR¹¹R¹¹—CAP, Link —(CH₂)ₙ—NR¹³—CH₂(CHOR⁸)(CHOR⁸)ₙ—CR¹¹R¹¹—CAP, Link —(CH₂)ₙ—(CHOR⁸)ₘCH₂—NR¹³—(Z)_{g}—CR¹¹R¹¹—CAP, Link —(CH₂)ₙNR¹³—(CH₂)ₘ(CHOR^{B})ₙCH₂NR¹³—(Z)_{g}—CR¹¹R¹¹—CAP, Link —(CH₂)ₘ—(Z)_{g}—(CH₂)ₘ—CR¹¹R¹¹—CAP, Link NH—C(═O)—NH—(CH₂)ₘ—CR¹¹R¹¹—CAP, Link —(CH₂)ₘ—C(═O)NR¹³—(CH₂)ₘ—CR¹¹R¹¹—CAP, Link —(CH₂)ₙ—(Z)_{g}—(CH₂)ₘ—(Z)_{g}—CR¹¹R¹¹—CAP, Link —Z_{g}—(CH₂)ₘ—CR¹¹R¹¹—CAP,

wherein Link is, independently, —O—, (CH₂)_{n—, —O(CH}₂)ₘ—, —NR¹³—C(═O)—NR¹³, —NR¹³—C(═O)—(CH₂)ₘ—, —C(═O)NR¹³—(CH₂)ₘ, —(CH₂)ₙ—Z_{g}—(CH₂)ₙ, —SO—, —SO₂—, SO₂NR⁷—, SO₂NR¹⁰—, -Het-.

wherein each CAP is, independently,

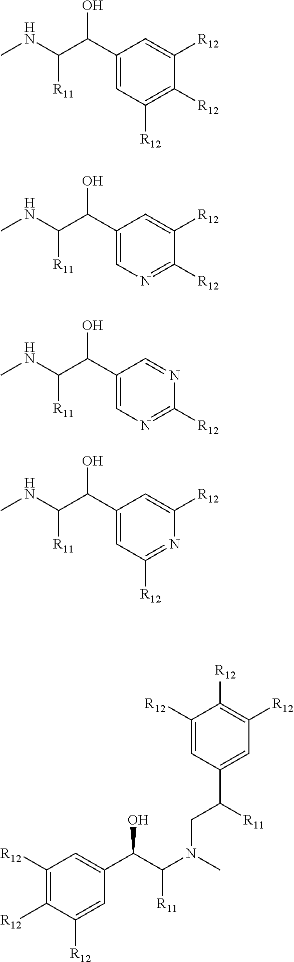

each R⁶ is, independently, —R⁷, —OR⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R ¹⁰, —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₙ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

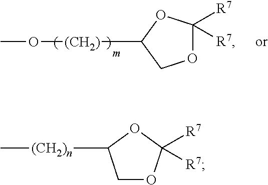

where when two R⁶ are —OR¹¹ are located adjacent to each other on a phenyl ring, the alkyl moieties of the two R⁶ may be bonded together to form a methylenedioxy group; with the proviso that when at least two —CH₂OR⁸ are located adjacent to each other, the R⁸ groups may be joined to form a cyclic mono- or di-substituted 1,3-dioxane or 1,3-dioxolane,

each R⁷ is, independently, hydrogen, lower alkyl, phenyl, or substituted phenyl;

each R⁸ is, independently, hydrogen, lower alkyl, —C(═O)—R¹¹, glucuronide, 2-tetrahydropyranyl, or

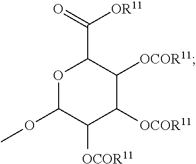

each R⁹ is, independently, CO₂R¹³, —CON(R¹³)₂, —SO₂CH₂R¹³, or —C(═O)R¹³;

each R¹⁰ is, independently, —H, —SO₂CH₃, —CO₂R⁷, —C(═O)NR⁷R⁹, —C(═O)R⁷, or —(CH₂)ₘ—(CHOH)ₙ—CH₂OH;

each Z is, independently, CHOH, C(═O), —(CH₂)ₙ—, CHNR¹³R¹³, C═NR¹³, or NR¹³; each R¹¹ is, independently, hydrogen, lower alkyl, phenyl lower alkyl or substituted phenyl lower alkyl;

each R¹² is independently, —(CH₂)ₙ—SO₂CH₃, —(CH₂)ₙ—CO₂R¹³, —(CH₂)ₙ—C(═O)NR¹³R¹³, —(CH₂)ₙ—C(═O)R¹³, —(CH₂)ₙ—(CHOH)ₙ—CH₂OH, —NH—(CH₂)ₙ—SO₂CH₃, NH—(CH₂)ₙ—C(═O)R¹¹, NH—C(═O)—NH—C(═O)R¹¹, —C(═O)NR¹³R¹³, —OR¹¹, —NH—(CH₂)ₙ—R¹⁰, —Br, —Cl, —F, —I, SO₂NHR¹¹, —NHR¹³, —NH—C(═O)—NR¹³R¹³, NH—(CH₂)ₙSO₂CH₃, NH—(CH₂)ₙ—C(═O)R¹¹, —NH—C(═O)—NH—C(═O)R¹¹, —C(═O)NR¹³R¹³, —OR¹¹, —(CH₂)ₙ—NHR¹³, —NH—C(═O)—NR¹³R¹³, or —NH—(CH₂)ₙ—C(═O)—R¹³;

each R¹³ is, independently, hydrogen, lower alkyl, phenyl, substituted phenyl,

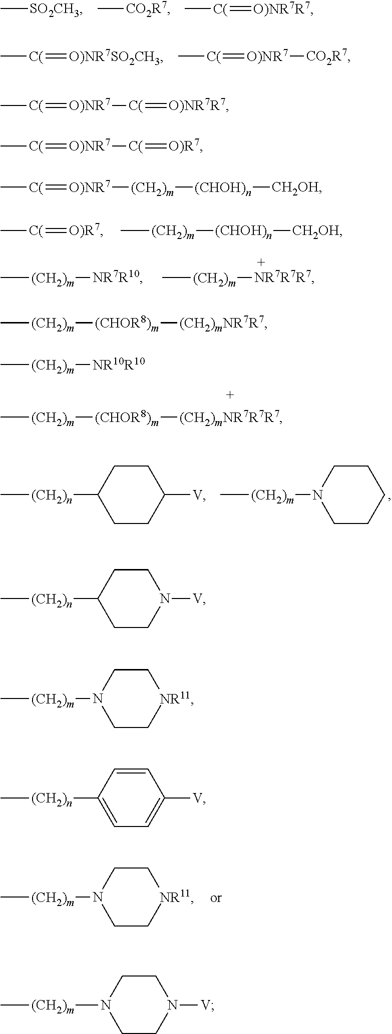

with the proviso that NR¹³R¹³ can be joined on itself oi a group represented by one of the following:

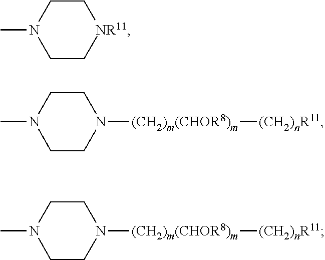

each Het is independently, —NR¹³, —S—, —SO—, —SO₂—, —O—, —SO₂NR¹³—, —NHSO₂—, —NR¹³CO—, or —CONR¹³—;

each g is, independently, an integer from 1 to 6;

each m is, independently, an integer from 1 to 7;

each n is, independently, an integer from 0 to 7;

each Q′ is, independently, CR⁶ or N;

each Q is independently, —C(R⁶R⁵)—, —C(R⁶R⁶)—, —N(R¹⁰)—, —N(R⁷)—, —N(R⁵)—, —S—, —SO—, or —SO₂—,

with the proviso that at least one Q is —C(R⁵R⁶)— or —N(R⁵)—,

with the proviso that at most three Q in a ring is —N(R⁷)—, —N(R⁵)—, —S—, —SO—, or —SO₂—;

each V is, independently,

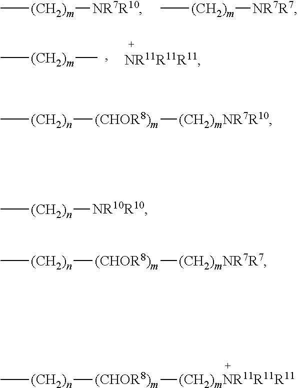

with the proviso that when V is attached directly to a nitrogen a th n V an also be, independently, R⁷, R¹⁰, or (R¹¹)₂;

wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane,

wherein any of the above compounds can be a pharmaceutically acceptable salt thereof, and wherein the above compounds are inclusive of all racemates, enantiomers, diastereomers, tautomers, polymorphs and pseudopolymorphs thereof.

The present invention also provides a method of promoting mucus clearance in mucosal surfaces, comprising:

administering an effective amount of a compound represented by formula (I) to a mucosal surface of a subject.

The present invention also provides a method of treating chronic bronchitis, comprising:

administering an effective amount of a compound represented by formula (I) to a subject in need thereof.

The present invention also provides a method of treating cystic fibrosis, comprising:

administering an effective amount of compound represented by formula (I) to a subject in need thereof.

The present invention also provides a method of treating rhinosinusitis, comprising:

administering an effective amount of a compound represented by a formula (I) to a subject in need thereof.

The present invention also provides a method of treating nasal dehydration, comprising:

administering an effective amount of a compound represented by formula (I) to the nasal passages of a subject in need thereof.

In a specific embodiment, the nasal dehydration is brought on by administering dry oxygen to the subject.

The present invention also provides a method of treating sinusitis, comprising:

administering an effective amount of a compound represented by formula (I) to a subject in need thereof.

The present invention also provides a method of treating pneumonia, comprising:

administering an effective amount of a compound represented by formula (I) to a subject in need thereof.

The present invention also provides a method of preventing ventilator-induced pneumonia, comprising:

administering an effective compound represented by formula (I) to a subject by means of a ventilator.

The present invention also provides a method of treating asthma, comprising:

administering an effective amount of a compound represented by formula (I) to a subject in need thereof.

The present invention also provides a method of treating primary ciliary dyskinesia, comprising:

administering an effective amount of a compound represented by formula (I) to a subject in need thereof.

The present invention also provides a method of treating otitis media, comprising:

administering an effective amount of a compound represented by formula (I) to a subject in need thereof.

The present invention also provides a method of inducing sputum for diagnostic purposes, comprising:

administering an effective amount of compound represented by formula (I) to a subject in need thereof.

The present invention also provides a method of treating chronic obstructive pulmonary disease, comprising:

administering an effective amount of a compound represented by formula (I) to a subject in need thereof.

The present invention also provides a method of treating emphysema, comprising:

administering an effective amount of a compound represented by formula (I) to a subject in need thereof.

The present invention also provides a method of treating dry eye, comprising:

administering an effective amount of a compound represented by formula (I) to the eye of the subject in need thereof.

The present invention also provides a method of promoting ocular hydration, comprising:

administering an effective amount of a compound represented by formula (I) to the eye of the subject.

The present invention also provides a method of promoting corneal hydration, comprising:

administering an effective amount of a compound represented by formula (I) to the eye of the subject.

The present invention also provides a method of treating Sjögren's disease, comprising:

administering an effective amount of compound represented by formula (I) to a subject in need thereof.

The present invention also provides a method of treating vaginal dryness, comprising:

administering an effective amount of a compound represented by formula (I) to the vaginal tract of a subject in need thereof.

The present invention also provides a method of treating dry skin, comprising:

administering an effective amount of a compound represented by formula (I) to the skin of a subject in need thereof.

The present invention also provides a method of dry mouth (xerostomia), comprising:

administering an effective amount of compound represented by formula (I) to the mouth of the subject in need thereof.

The present invention also provides a method of treating distal intestinal obstruction syndrome, comprising:

administering an effective amount of compound represented by formula (I) to a subject in need thereof.

The present invention also provides a method of treating esophagitis, comprising:

administering an effective amount of a compound represented by formula (I) to a subject in need thereof.

The present invention also provides a method of treating constipation, comprising:

administering an effective amount of a compound represented by formula (I) to a subject in need thereof. In one embodiment of this method, the compound is administered either orally or via a suppository or enema.

The present invention also provides a method of treating chronic diverticulitis comprising:

administering an effective amount of a compound represented by formula (I) to a subject in need thereof.

### BRIEF DESCRIPTION OF THE FIGURES

FIG. 1 shows the baseline activity of sodium channels before and after blockade with amiloride.

FIG. 2 shows the activity of sodium channels before and after the addition of a beta-agonist

FIG. 3 shows the mechanism underlying the additivity of a Na channel blocker and a beta-agonist.

FIG. 4 shows the tautomers of the compounds of formula I.

### DETAILED DESCRIPTION OF THE INVENTION

The present invention is based on the discovery that the compounds of formula (I) also possess both sodium channel blocking activity and beta agonist activity in the same molecule.

The present invention is also based on the discovery that the compounds of formula (I) are more potent and/or, absorbed less rapidly from mucosal surfaces, especially airway surfaces, and/or less reversible from interactions with ENaC as compared to compounds such as amiloride, benzarnil, and phenamil. Therefore, the compounds of formula (I) have a longer half-life on mucosal surfaces as compared to these compounds.

The present invention is also based on the discovery that certain compounds embraced by formula (I) are converted in vivo into metabolic derivatives thereof that have reduced efficacy in blocking sodium channels and acting as beta-adrenergic receptor agonists as compared to the parent administered compound, after they are absorbed from mucosal surfaces after administration. This important property means that the compounds will have a lower tendency to cause undesired side-effects by blocking sodium channels and activating beta-receptors located at other untargeted locations in the body of the recipient, e.g., in the kidneys and heart.

Mono drug therapy leaves most major diseases such as chronic bronchitis and cystic fibrosis inadequately treated. It is therefore often necessary to discover and develop novel drugs or combination of drugs which treat and modulate multiple targets simultaneously (polypharmacology) with the goal of enhancing efficacy or improving safety relative to single target drugs. There are three possible ways to achieve this. 1) Combining therapeutic “cocktails” of two or more individual drugs; the benefits of this approach are often lessened by poor patient compliance. 2). A multiple component drug (“fixed combination” or multiple component drug) that contains two or more agents in a single tablet, liquid formulation, inhaler or dry powder device. This can sometimes improve patient compliance versus multiple component drugs but adds the complexity of carefully dosing so as to minimize multiple metabolic pathways. 3). A single molecular entity which can simultaneously modulate multiple drug targets (designed multiple ligands). The advantage of a multiple ligand over the first two approaches is that it improves compliance, enhances efficacy, it targets a known set of deficiencies in multiple systems with a single new chemical entity, it often lacks the unpredictable differences in the pharmacokinetic and pharmacodynamic variability between patients, it is often easier to formulate and potentially lowers the risk of drug-drug interactions compared to drug cocktails and multiple component drugs. It was therefore our goal to discover multiple ligands that have both sodium channel blocking activity as well as beta agonist activity.

The addition of beta-adreneraic receptor agonist activity to a sodium channel blocker will significantly increase the capacity to hydrate airway surfaces in subjects in need of hydration for therapeutic purposes. The mechanism by which beta-agonist activity adds to the hydration capacity of Na channel blockers alone, or beta-agonists alone, is described in the following diagrams that describe the electrochemical gradients for ion flows and the net secretion that results from these forces in airway epithelia.

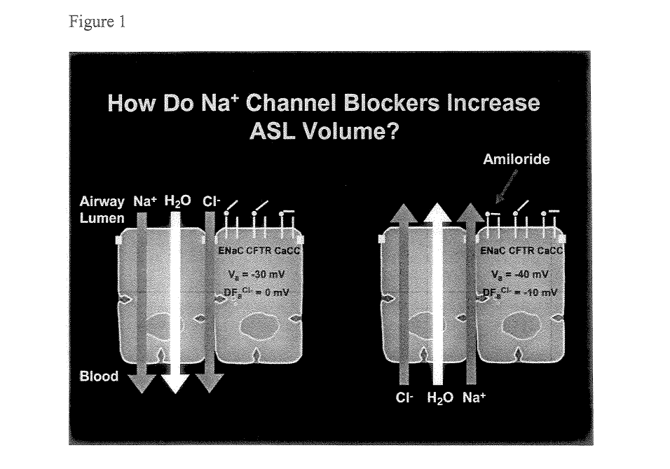

As shown in FIG. 1, under baseline conditions human airway epithelia absorb NaCl and H₂O. Active Na⁺ absorption drives this process. Cl⁻ is absorbed passively with Na⁺ to preserve electroneutrality. As there is no net driving force for Cl⁻ to move across the apical cell membrane, Cl⁻ is absorbed paracellularly in response to the transepithelial electric potential. Water moves cellularly and paracellularly in reponse to the osmotic gradients generated by NaCl absorbtion.

Application of a Na⁺ channel blocker (as an example amiloride is shown) inhibits the entry of Na⁺ into the cell which: (1) abolishes Na⁺ absorption and (2) hyperpolarizes the apical cell membrane (Va). The hyperpolarization of Va generates an electrochemical driving force favoring cr secretion (Na⁺ now follows in the secretory direction via the paracellularpath). The rate of Cl⁻ secretion is proportional to the activity of the apicalmembrane Cl⁻ channels which are typically 30-50% maximally active under basal conditions. In summary, application of a Na⁺ channel blocker inhibits Na⁺ absorption and triggers a modest amount of Cl⁻ secretion. Note again that water will follow transcellularly in response to the secreted NaCl.

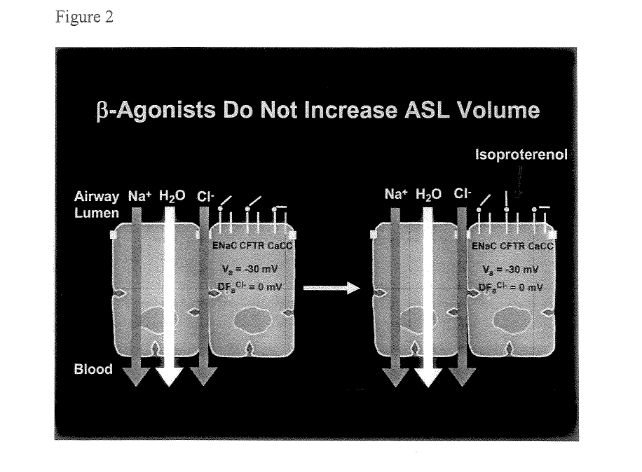

In contrast, as depicted in FIG. 2, addition of a beta-agonist (as an example isoproterenol is shown) alone to human airway epithelia produces no changes in Na⁺ absorption or Cl⁻ secretion. The reason for this absence of effect is that there is no electrochemical driving force for Cl⁻ to move across the cell (See the following references: Intracellular Cl— activityand cellular Cl— pathways in cultured human airway epithelium. Am J Physiol. 1989 May ;256(5 Pt 1):C1033-44. Willumsen N J, Davis C W, Boucher R C Cellular Cl— transport in cultured cystic fibrosis airway epithelium. Am Physiol. 1989 May ;256(5 Pt 1):C1045-53. Willumsen N J, Davis C W, Boucher R C Activation of an apical Cl— conductance by Ca2+ ionophores in cystic fibrosis airway epithelia. Am Physiol. 1989 February ;256(2 Pt 1):C226-33. Willumsen N J, Boucher R C). Thus, a beta-agonist mediated activation of an apical membrane Cl⁻ channel, usually CFTR via changes in cAMP, produces no change in the rate of movement of Cl⁻ across the barrier and, hence, no change in transepithelial sodium chloride or water secretion.

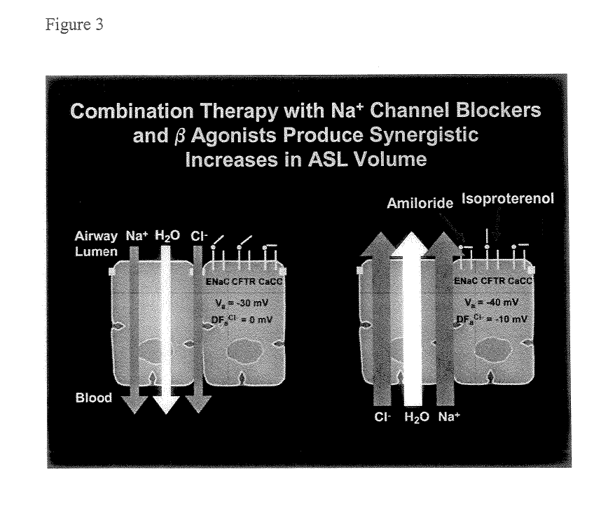

However, when a Na channel blocker is administered with a beta-agonist, additivity between these two classes of compounds is achieved with the result beine accelerated Cl⁻ (and Na⁺, H₂O) secretion. The mechanism underlying the additivity is shown in FIG. 3. In the presence of a Na channel blocker, an electrochemical gradient for Cl⁻ secretion is generate (also see FIG. 1). Now when a beta-agonist is present, it converts the apical membrane CFTR from ˜30% basal activity to ˜100% activity via beta-agonist induced increase in cAMP that ultimately activates CFTR via PKA (protein kinase A). Because there is an electrochemical driving force favoring Cl⁻ secretion as a result of ENaC blockade, the increase in Cl⁻ channel activity translates into increasing Cl⁻ (and Na⁺, H₂O) secretion. Thus, the hydration capacity of the epithelia is greatly enhanced by the presence of both Na⁺ channel blocker and beta-adrenereic receptor agonist activities in the environment bathing the human airway epithelia as compared to just Na⁺ channel blocker or beta-adrenergic receptor agonist by themselves. A discovery of this invention is that administration of both activities contained within the same molecule to the epithelium is at least as effective as sequential administration of a Na channel blocker followed by a beta-agonist and therefore has the advantages cited earlier.

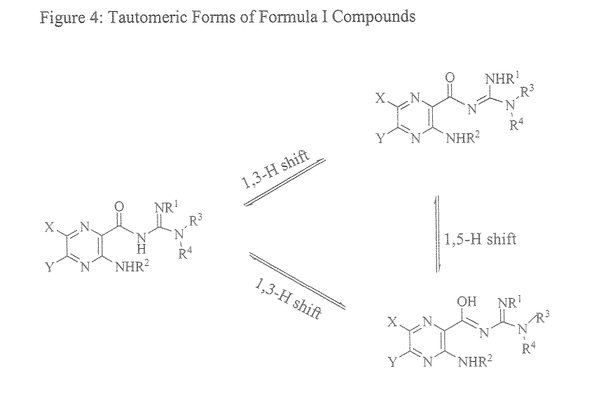

The compounds of formula I exist primarily as a combination of the three tautomers shown in FIG. 4. The compounds of formula I exist primarily as a combination of the three tautomers. FIG. 4 shows the three tantomers represented in formula I that exist in solution. Previous studies by Smith et al. have shown that the free base exists primarily as the acylimino tautomer, whereas the physiologically active species exists as the protonated form of the acylamino tautomer (FIG. 1, ref R L Smith et. Al, Journal of the American Chemical Society, 1979, 101, 191-201). These structural representations have been used to represent amiloride and its analogs in both the patent and scientific literature. We use both the acylamino and acylimino representations for convenience throughout this patent with the understanding that the structures are in reality a hybrid of the three forms with the actual amount of each dependent on the pH, the cite of action and the nature of the subs(ituents.

In the compounds represented by formula (I), X may be hydrogen, halogen, trifluoromethyl, lower alkyl, lower cycloalkyl, unsubstituted or substituted phenyl, lower alkyl-thio, phenyl-lower alkyl-thio, lower alkyl-sulfonyl, or phenyl-lower alkyl-sulfonyl. Halogen is preferred,

Examples of halogen include fluorine, chlorine, bromine, and iodine. Chlorine and bromine are the preferred halogens. Chlorine is particularly preferred. This description is applicable to the term “halogen” as used throughout the present disclosure.

As used herein, the term “lower alkyl” means an alkyl group having less than 8 carbon atoms. This range includes all specific values of carbon atoms and subranges there between, such as 1, 2, 3, 4, 5, 6, and 7 carbon atoms, The term “alkyl” embraces all types of such groups, e.g., linear, branched, and cyclic alkyl groups. This description is applicable to the term “lower alkyl” as used throughout the present disclosure. Examples of suitable lower alkyl groups include methyl, ethyl, propyl, cyclopropyl, butyl, isobutyl, etc.

Substituents for the phenyl group include halogens. Particularly preferred halogen substituents are chlorine and bromine.

Y may be hydrogen, hydroxyl, mercapto, lower alkoxy, lower alkyl-thio, halogen, lower alkyl, lower cycloalkyl, mononuclear aryl, or —N(R²)₂. The alkyl moiety of the lower alkoxy groups is the same as described above. Examples of mononuclear aryl include phenyl groups. The phenyl group may be unsubstituted or substituted as described above. The preferred identity of Y is —N(R²)₂. Particularly preferred are such compounds where each R² is hydrogen.

R¹ may be hydrogen or lower alkyl. Hydrogen is preferred for R¹.

Each R² may be, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, (CH₂)ₙ—C(═O)NR⁷R¹⁰, —(CH₂)ₙ—Z_{g}—R⁷, —(CH₂)ₘ—NR¹⁰—CH₂(CHOR⁸)(CHOR⁸)ₙ—CH₂OR⁸—(CH₂)ₙ—CO₂R⁷, or

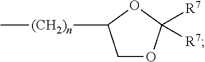

Hydrogen and lower alkyl, particularly C₁-C₃ alkyl are preferred for R². Hydrogen is particularly preferred.

R³ and R⁴ may be, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, provided that at least one of R³ and R⁴ is a group represented by formula (A).

Preferred compounds are those where one of R³ and R⁴ is hydrogen and the other is represented by formula (A).

In formula (A), the moiety —(C(R^{L})₂)ₒ-x-(C(R^{L})₂)ₚ— defines an alkylene group bonded to the aromatic ring. The variables o and p may each be an integer from 0 to 10, subject to the proviso that the sum of o and p in the chain is from 1 to 10, Thus, o and p may each be 0, 1, 2, 3 4, 5, 6, 7, 8, 9, or 10. Preferably, the sum of o and p is from 2 to 6. In a particularly preferred embodiment, the sum of o and p is 4.

The linking group in the alkylene chain, x, may be, independently, O, NR¹⁰, C(═O), CHOH, C(═N—R¹⁰), CHNR⁷R¹⁰, or represents a single bond;

Therefore, when x represents a single bond, the alkylene chain bonded to the ring is represented by the formula —(C(R^{L})₂)ₒ₊ₚ—, in which the sum o+p is from 1 to 10.

Each R^{L} may be, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰), —(CH₂)ₙ—C(═O)NR⁷R¹⁰), —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

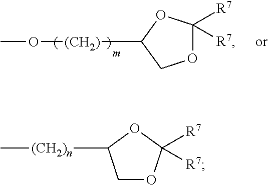

The preferred groups include —H, —OH, —N(R⁷)₂, especially where ach R⁷ is hydrogen.

In the alkylene chain in formula (A), it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, where in the other R^{L} groups in the chain are hydrogens. Even more preferably, only one R^{L} group in an alkylene chain is other than hydrogen, where in the other R^{L} groups in the chain are hydrogens. In these embodiments, it is preferable that x represents a single bond.

In another particular embodiment of the invention, all of the R^{L} groups in the alkylene chain are hydrogen. In these embodiments, the alkylene chain is represented by the formula —(CH₂)ₒ-x-(CH₂)ₚ—.

Each R⁵ is, independently,

Link —(CH₂)ₙ—CR¹¹R¹¹—CAP, Link —(CH₂)ₙ(CHOR⁸)(CHOR⁸)ₙ—CR¹¹R¹¹—CAP, Link —(CH₂CH₂O)ₘ—CH₂—CR¹¹R¹¹—CAP, Link —(CH₂CH₂O)ₘ—CH₂CH₂—CR¹¹R¹¹—CAP, Link —(CH₂)ₙ—(Z)_{g}—CR¹¹R¹¹—CAP, Link —(CH₂)ₙ(Z)_{g}—(CH₂)ₘ—CR¹¹R¹¹—CAP, Link —(CH₂)ₙ—NR¹³—CH₂(CHOR⁸)(CHOR⁸)ₙ—CR¹¹R¹¹—CAP, Link —(CH₂)ₙ—(CHOR⁸)ₘCH₂—NR¹³—(Z)_{g}—CR¹¹R¹¹—CAP, Link —(CH₂)ₙNR¹³—(CH₂)_{(CHOR}⁸)ₙCH₂NR¹³—(Z)_{g}—CR¹¹R¹¹—CAP, Link —(CH₂)ₘ—(Z)_{g}—(CH₂)ₘ—CR¹¹R¹¹—CAP, Link NH—C(═O)—NH—(CH₂)ₘCR¹¹R¹¹—CAP, Link —(CH₂)ₘ—C(═O)NR¹³—(CH₂)ₘ—CR¹¹R¹¹—CAP, Link —(CH₂)ₙ—(Z)_{g}—(CH₂)ₘ—(Z)_{g}—CR¹¹R¹¹—CAP, or Link —Z_{g}—(CH₂)ₘ-Het-(CH₂)ₘ—CR¹¹R¹¹—CAP.

Each Link is, independently

—O—, (CH₂)ₙ—, —O(CH₂)ₘ—, —NR¹³—C(═O)—NR¹³, —NR¹³C(═O)—(CH₂)ₘ—, —C(═O)NR¹³—(CH₂)ₘ, —(CH₂)ₙ—Z_{g}—(CH₂)ₙ, —S—, —SO—, —SO₂—, SO₂NR⁷—, SO₂NR¹⁰—, or -Het-,

Each CAP is, dependently,

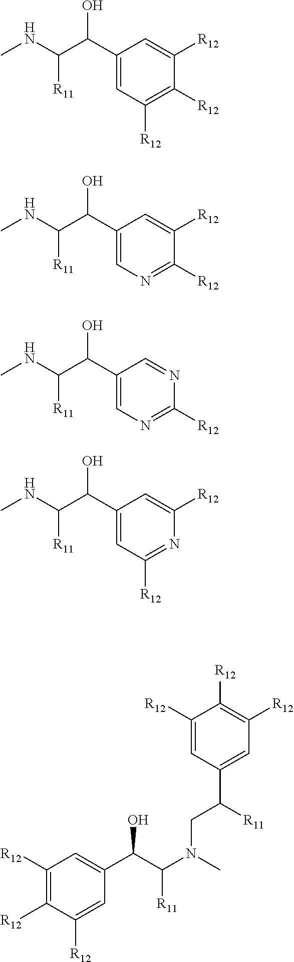

Each R⁶ is, independently, —R⁷, —OR⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

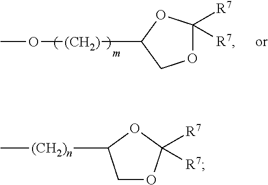

where when two R⁶ are —OR¹¹ and are located adjacent to each other on a phenyl ring, the alkyl moieties of the two R⁶ may be bonded together to form a methylenedioxy group; with the proviso that when at least two —CH₂OR⁸ are located adjacent to each other, the R⁸ groups may be joined to form a cyclic mono- or di-substituted 1,3-dioxane or 1,3-dioxolane,

Each R⁷ is, independently, hydrogen lower alkyl, phenyl, substituted phenyl or —CH₂(CHOR)⁸ₘ—R¹⁰.

Each R⁸ is, independently, hydrogen, lower alkyl, —C(═O)—R¹¹, glucuronide, 2-tetrahydropyranyl, or

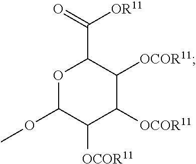

Each R⁹ is, independently, —CO₂R¹³, —CON(R¹³)₂, —SO₂CH₂R¹³, or —C(═O)R¹³.

Each R¹⁰ is, independently, —H, —SO₂CH₃, —CO₂R⁷, —C(═O)NR⁷R⁹, —C(═O)R⁷, or —(CH₂)ₘ—(CHOH)ₙ—CH₂OH.

Each Z is, independently, CHOH, C(═O), —(CH₂)ₙ—, CHNR¹³R¹³, C═NR¹³, or NR¹³.

Each R¹¹ is, independently, hydrogen, lower alkyl, phenyl lower alkyl or substituted phenyl lower alkyl.

Each R¹² is independently, —(CH₂)ₙ—SO₂CH₃, —(CH₂)ₙ—CO₂R¹³, —(CH₂)ₙ—C(═O)NR¹³R¹³, —(CH₂)ₙ—C(═O)R¹³, —(CH₂)ₙ—(CHOH)ₙ—CH₂OH, —NH—(CH₂)ₙ—SO₂CH₃, NH—(CH₂)ₙ—C(═O)R¹¹, NH—C(═O)—NH—C(═O)R¹¹, —C(═O)NR¹³R¹³, —OR¹¹, —NH—(CH₂)ₙ—R¹⁰, —Br, —Cl, —F, SO₂NHR¹¹, —NH R¹³, —NH—C(═O)—NR¹³R¹³, NH—(CH₂)ₙ—SO₂CH₃, NH—(CH₂)ₙ—C(═O)R¹¹, —NH—C(═O)—NH—C(═O)R¹¹, —C(═O)NR¹³R¹³, —OR¹¹, —(CH₂)ₙ—NHR¹³, —NH—C(═O)—NR¹³R¹³, or —NH—(CH₂)ₙ—C(═O)—R¹³.

Each R¹³ is, independently, hydrogen, lower alkyl, phenyl, substituted phenyl,

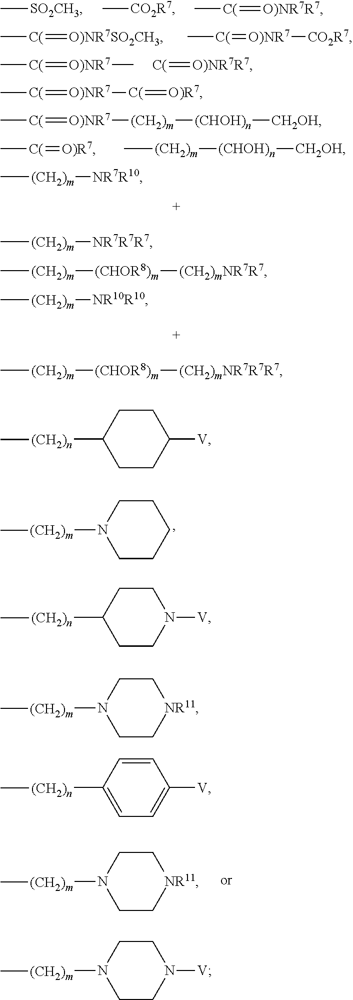

with the proviso that NR¹³R¹³ can be joined on itself to fo a group represented by one of the following:

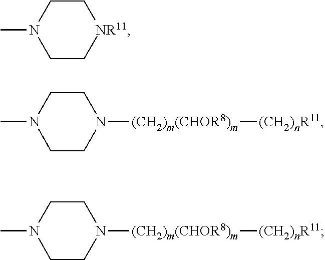

Each Het is independently, —NR¹³, —S—, —SO—, SO₂—, —O—, —SO₂NR¹³—, —NHSO₂—, —NR¹³CO—, or —CONR¹³—.

Each g is, independently, an integer from 1 to 6.

Each m is, independently, an integer from 1 to 7.

Each n is, independently, an integer from 0 to 7.

Each Q′ is, independently, CR⁶ or N. In one embodiment. Q′ is CH,

Each Q is independently, —C(R⁶R⁵)—, —C(R⁶R⁶)—, —N(R¹⁰)—, —N(R⁷)—, —N(R⁵)—. —S—, —SO—, or —SO₂—. At least one Q is —C(R⁵R⁶)— or —N(R⁵)—. In a preferred embodiment, at least one Q is —CHR⁵. In a particularly preferred embodiment each —C(R⁵R⁶) is —CHR⁶. At most three Q in a ring is —N(R⁷)—, —N(R⁵)—, —S—, —SO—, or —SO₂—. In a preferred embodiment, one or two Q is —N(R⁷)—, —N(R⁵)—, —S—, —SO—, or —SO₂-. In another embodiment, none of the Qs are —N(R⁷)—, —N(R⁵)—, —S—, —SO—, or —SO₂—, each Q is —C(R⁵R⁶).

Each V is, independently,

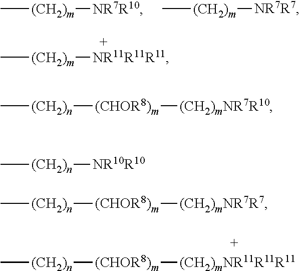

with the proviso that when V is attached directly to a nitrogen atom, then V can also be, independently, R⁷, R¹⁰, or (R¹¹)₂.

In one embodiment of the invention, when two —CH₂OR⁸ groups are located 1,2- or 1,3- with respect to each other the R⁸ groups may be joined to form a cyclic mono- or di-substituted 1,3-dioxane or 1,3-dioxolane.

In another embodiment, when two R⁶ are —OR¹¹ and are located adjacent to each other on a phenyl ring, the alkyl moieties of the two R⁶ may be bonded together to form a methylenedioxy group.

In another embodiment, when at least two —CH₂OR⁸ are located adjacent to each other, the R⁸ groups may be joined to form a cyclic mono- or di-substituted 1,3-dioxane or 1,3-dioxolane.

In addition, one of more of the R⁶ groups can be one of the R⁵ groups which fall within the broad definition of R⁶ set forth above.

When two R⁶ are —OR¹¹ and are located adjacent to each other on a phenyl ring, the alkyl moieties of the two R⁶ groups may be bonded together to form a methylenedioxy group, i.e., a group of the formula —O—CH₂—O—.

As discussed above, R⁶ may be hydrogen. Therefore, 1, 2, 3, or 4 R⁶ groups may be other than hydrogen. Preferably at most 3 of the R⁶ groups are other than hydrogen.

Each g is, independently, an integer from 1 to 6. Therefore, each g may be 1, 2, 3, 4, 5, or 6.

Each m is an integer from 1 to 7, Therefore, each m may be 1, 2, 3, 4, 5, 6, or 7.

Each n is an integer from 0 to 7, Therefore, each n may be 0, 1, 2, 3, 4, 5, 6, or 7.

A more specific embodiment of a group represented by formula (A) is one in which each R^{L} is hydrogen and Q′ is CH.

In a preferred embodiment of the invention, Y is —NH₂.

In another preferred embodiment, R² is hydrogen,

In another preferred embodiment, R¹ is hydrogen.

In another preferred embodiment, X is chlorine,

In another preferred embodiment, R³ is hydrogen.

In another preferred embodiment, R^{L} is hydrogen.

In another preferred embodiment, o is 4.

In another preferred embodiment, p is 0.

In another preferred embodiment, the sum of o and p is 4.

In another preferred embodiment, x represents a sinde bond.

In another preferred embodiment, R⁶ is hydrogen.

In another preferred embodiment, at most one Q in a ring are —N(R⁷)—, —N(R⁵)—, —S—, —SO—, or —SO₂—. In another preferred embodiment, no Q is —N(R⁷)—, —N(R⁵)—, —S—, —SO—, or —SO₂—.

In a preferred embodiment of the present invention:

X is halogen;

Y is —N(R⁷)₂;

R¹ is hydrogen or C₁-C₃ alkyl;

R² is —R⁷, —OR⁷, CH₂OR⁷, or —CO₂R⁷;

R³ is a group represented by formula (A); and

R⁴ is hydrogen, a group represented by formula (A), or lower alkyl;

In another preferred embodiment of the present invention:

X is chloro or bromo;

Y is —N(R⁷)₂;

R² is hydrogen or C₁-C₃ alkyl;

at most three R⁶ are other than hydrogen as described above;

at most three R^{L} are other than hydrogen as described above; and

at most 2 Q in a ring are —N(R⁷)—, —N(R⁵)—, —S—, —SO—, or —SO₂—.

In another preferred embodiment of the present invention:

Y is —NH₂;

In another preferred embodiment of the present invention:

R⁴ is hydrogen;

at most one R^{L} is other than hydrogen as described above;

at most two R⁶ are other than hydrogen as described above; and

at most 1 Q in a ring is —N(R⁷)—, —N(R⁵)—, —S—. —SO—, or —SO₂—.

Specific examples of compounds within the scope of the invention are:

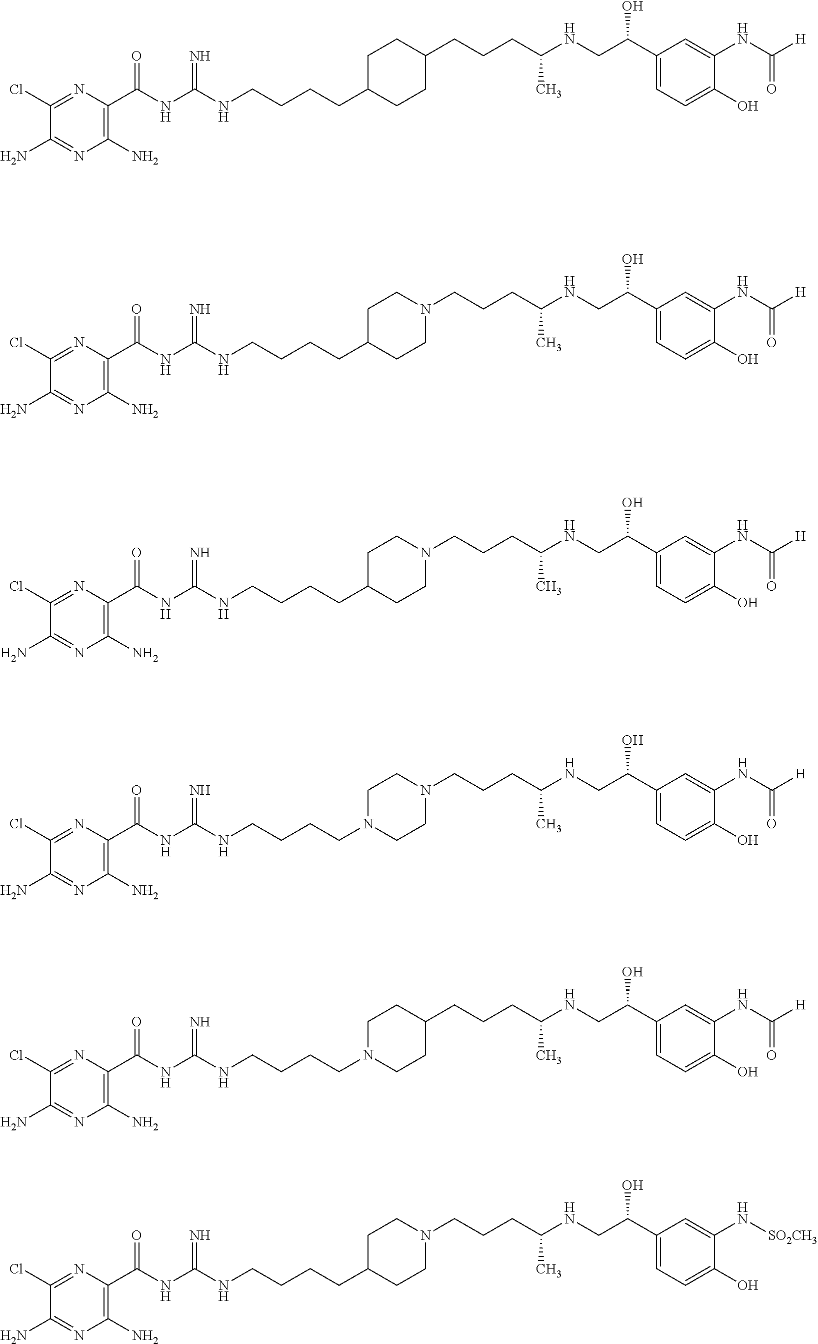

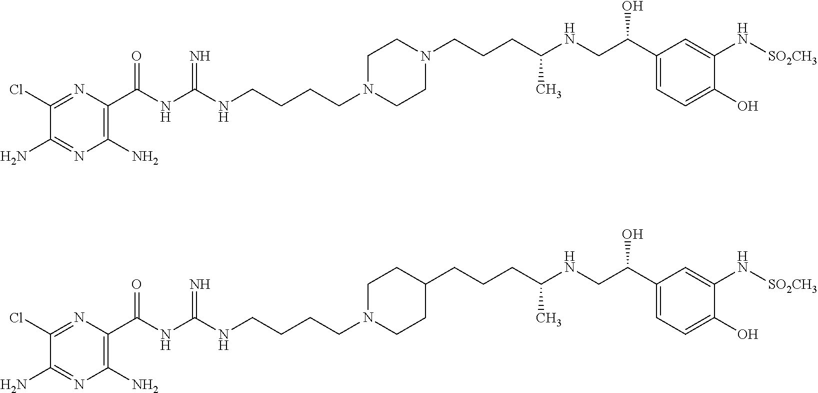

The compounds of formula (I) may be prepared and used as the free base. Alternatively, the compounds may be prepared and used as a pharmaceutically acceptable salt. Pharmaceutically acceptable salts are salts that retain or enhance the desired biological activity of the parent compound and do not impart undesired toxicological effects, Examples of such salts are (a) acid addition salts formed with inorganic acids, for example, hydrochloric acid, hydrobromic acid, sulfuric acid, phosphoric acid, nitric acid and the like; (b) salts formed with organic acids such as, for example, acetic acid, oxalic acid, tartaric acid, succinic acid, maleic acid, fumaric acid, gluconic acid, citric acid, mane acid, ascorbic acid, benzoic acid, tannic acid, palmi lc acid, alginic acid, polyglutamic acid, naphthalenesulfonic acid, methanesuifonic acid, p-toluenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid and the like; and (c) salts formed from elemental anions for example, chlorine, bromine, and iodine.

It is to be noted that all enantiomers, diastereomers, tautomers and racemic mixtures of compounds within the scope of formula (I) are embraced by the present invention. All mixtures of such enantiomers and diastereomers are within the scope of the present invention.

Without being limited to any particular theory, it is believed that the compounds of formula (I) function in vivo as sodium channel blockers and as beta receptor agonists. By blocking epithelial sodium channels as well as activating beta-receptors present in mucosal surfaces the compounds of formula (I) reduce the absorption of water by the mucosal surfaces. This effect increases the volume of protective liquids on mucosal surfaces, rebalances the system, and thus treats disease.

The present invention also provides methods of treatment that take advantage of the properties of the compounds of formula (I) discussed above. Thus, subjects that may be treated by the methods of the present invention include, but are not limited to, patients afflicted with cystic fibrosis, primary ciliary dyskinesia, chronic bronchitis, chronic obstructive airway disease, artificially ventilated patients, patients with acute pneumonia, etc. The present invention may be used to obtain a sputum sample from a patient by administering the active compounds to at least one lung of a patient, and then inducing or collecting a sputum sample from that patient. Typically, the invention will be administered to respiratory mucosal surfaces via aerosol (liquid or dry powders) or lavage.

Subjects that may be treated by the method of the present invention also include patients being administered supplemental oxygen nasally (a regimen that tends to dry the airway surfaces); patients afflicted with an allergic disease or response (e.g., an allergic response to pollen, dust, animal hair or particles, insects or insect particles, etc.) that affects nasal airway surfaces; patients afflicted with a bacterial infection e.g., staphylococcus infections such as Staphylococcus aureus infections, Hemophilus influenza infections, Streptococcus pneumoniae infections, Pseudomonas aeuriginosa infections, etc.) of the nasal airway surfaces; patients afflicted with an inflammatory disease that affects nasal airway surfaces; or patients afflicted with sinusitis (wherein the active agent or agents are administered to promote drainage of congested mucous secretions in the sinuses by administering an amount effective to promote drainage of congested fluid in the sinuses), or combined, Rhinosinusitis, The invention may be administered to rhino-sinal surfaces by topical delivery, including aerosols and drops.

The present invention may be used to hydrate mucosal surfaces other than airway surfaces. Such other mucosal surfaces include gastrointestinal surfaces, oral surfaces, genito-urethral (vaginal) surfaces, ocular surfaces or surfaces of the eye, the inner ear and the middle ear. For example, the active compounds of the present invention may be administered by any suitable means, including locally/topically, orally, or rectally, in an effective amount.

The present invention is concerned primarily with the treatment of human subjects, but may also be employed for the treatment of other mammalian subjects, such as dogs and cats, for veterinary purposes.

As discussed above, the compounds used to prepare the compositions of the present invention may be in the form of a pharmaceutically acceptable free base. Because the free base of the compound is generally less soluble in aqueous solutions than the salt, free base compositions are employed to provide more sustained release of active agent to the lungs. An active agent present in the lungs in particulate form which has not dissolved into solution yet serves as a depot of drug which gradually becomes bioavailable as it slowly dissolves into solution.

Another aspect of the present invention is a pharmaceutical mposition, comprising a compound of formula (I) in a pharmaceutically acceptable carrier (e.g, an aqueous carrier solution). In general, the compound of formula (I) is included in the composition in an amount effective to inhibit the reabsorption of water by mucosal surfaces.

The compounds of the present invention may also be used in conjunction with a P2Y2 receptor agonist or a pharmaceutically acceptable salt thereof (also sometimes referred to as an “active agent” herein). The composition may further comprise a P2Y2 receptor agonist or a pharmaceutically acceptable salt thereof (also sometimes referred to as an “active agent” herein). The P2Y2 receptor agonist is typically included in an amount effective to stimulate chloride and water secretion by airway surfaces, particularly nasal airway surfaces. Suitable P2Y2 receptor agonists are described in columns 9-10 of U.S. Pat. No. 6,264,975, U.S. Pat. No. 5,656,256, and U.S. Pat. No. 5,292,498, each of which is incorporated herein by reference,

Bronchodilators can also be used in combination with compounds of the present invention. These bronchodilators include, but are not limited to, anticholinergic agents including but not limited to ipratropium bromide, as well as compounds such as theophylline and aminophylline. These compounds may be administered in accordance with known techniques, either prior to or concurrently with the active compounds described herein.

Ionic and organic osmolytes can also be used in combination with compounds of the present invention. Ionic osmolytes useful include any salt consisting of a pharmaceutically acceptable anion and a pharmaceutical cation. Organic osmolytes include, but are not limited to, sugars, sugar alcohols and organic osmolytes. Detailed examples of ionic and non-ionic osmolytes are given in U.S. Pat. No. 6,926,911, incoroprated herein by reference. A particularly useful ionic osmolyte is hypertonic sodium chloride or sodium nitrite. A particularly useful organic osmolyte is the reduced sugar mannitol.

Another aspect of the present invention is a pharmaceutical formulation, comprising an active compound as described above in a pharmaceutically acceptable carrier (e.g., an aqueous carrier solution). In general, the active compound is included in the composition in an amount effective to treat mucosal surfaces, such as inhibiting the reabsorntion of water by mucosal surfaces, including airway and other surfaces,

The active compounds disclosed herein may be administered to mucosal surfaces by any suitable means, including topically, orally, rectally, vaginally, ocularly and dermally, etc. For example, for the treatment of constipation, the active compounds may be administered orally or rectally to the gastrointestinal mucosal surface. The active compound may be combined with a pharmaceutically acceptable carrier in any suitable form, such as sterile physiological or dilute saline or topical solution, as a droplet, tablet or the like for oral administration, as a suppository for rectal o nito-urethral administration, etc. Excipients may be included in the formulation to enhance the solubility of the active compounds, as desired.

The active compounds disclosed herein may be administered to the airway surfaces of a patient by any suitable means, including as a spray, mist, or droplets of the active compounds in a pharmaceutically acceptable carrier such as physiological or dilute saline solutions or distilled water. For example, the active compounds may be prepared as formulations and administered as described in U.S. Pat. No. 5,789,391 to Jacobus, the disclosure of which is incorporated by reference herein in its entirety.

Solid or liquid particulate active agents prepared for practicing the present invention could, as noted above, include particles of respirable or non-respirable size; that is, for respirable particles, particles of a size sufficiently small to pass through the mouth and larynx upon inhalation and into the bronchi and alveoli of the lungs, and for non-respirable particles, particles sufficiently large to be retained in the nasal airway passages rather than pass through the larynx and into the bronchi and alveoli of the lungs. In general, particles ranging from about 1 to 5 microns in size (more particularly, less than about 4.7 microns in size) are respirable. Particles of non-respirable size are greater than about 5 microns in size, up to the size of visible droplets. Thus, for nasal administration, a particle size in the range of 10-500 μm may be used to ensure retention in the nasal cavity.

In the manufacture of a formulation according to the invention, active agents or the physiologically acceptable salts or free bases thereof are typically admixed with, inter alia, an acceptable carrier. Of course, the carrier must be compatible with any other ingredients in the formulation and must not be deleterious to the patient. The carrier must be solid or liquid, or both, and is preferably formulated with the compound as a unit-dose formulation, for example, a capsule, that may contain 0.5% to 99% by weight of the active compound. One or more active compounds may be incorporated in the formulations of the invention, which formulations may be prepared by any of the well-known techniques of pharmacy consisting essentially of admixing the components.

Compositions containing respirable or non-respirable dry particles of micronized active agent may be prepared by grinding the dry active agent with a mortar and pestle, and then passing the micronized composition through a 400 mesh screen to break up or separate out larae agglomerates.

The particulate active agent composition may optionally contain a dispersant which serves to facilitate the formulation of an aerosol. A suitable dispersant is lactose, which may be blended with the active agent in any suitable ratio (e.g., a 1 to 1 ratio by weight).

Active compounds disclosed herein may be administered to airway surfaces including the nasal passaaes, sinuses and lungs of a subject by a suitable means know in the art, such as by nose drops, mists, etc. In one embodiment of the invention, the active compounds of the present invention and administered by transbronchoscopic lavage. In a preferred embodiment of the invention, the active compounds of the present invention are deposited on lung airway surfaces by administering an aerosol suspension of respirable particles comprised of the active compound, which the subject inhales. The respirable particles may be liquid or solid. Numerous inhalers for administering aerosol particles to the lungs of a subject are known.

Inhalers such as those developed by Nolctar Therapeutic Systems, Palo Alto, Calif., USA, may be employed, including but not limited to those disclosed in U.S. Pat. Nos. 5,740,794; 5,654,007; 5,458,135; 5,775,320; and 5,785,049, each of which is incorporated herein by reference. The Applicant specifically intends that the disclosures of all patent references cited herein be incorporated by reference herein in their entirety. Inhalers such as those developed by Dura Pharmaceuticals, Inc., San Diego, Calif., USA, may also be employed, including but not limited to those disclosed in U.S. Pat. Nos. 5,622,166; 5,577,497; 5,645,051; and 5,492,112, each of which is incorporated herein by reference. Additionally, inhalers such as those developed by Aradigm Corp., Hayward, Calif., USA, may be employed, including but not limited to those disclosed in U.S. Pat. Nos. 5,826,570; 5,813,397; 5,819,726; and 5,655,516, each of which is incorporated herein by reference. These apparatuses are particularly suitable as dry particle inhalers.

Aerosols of liquid particles comprising the active compound may be produced by any suitable means, such as with a pressure-driven aerosol nebulizer or an ultrasonic nebulizer. See, e.g., U.S. Pat. No. 4,501,729, which is incorporated herein by reference. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or dilute aqueous alcoholic solution. Perfluorocarbon carriers may also be used. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing predetermined metered dose of medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of powder blend comprising the active ingredient, a suitable powder diluent, such as lactose, and an optional surfactant. The active ingredient typically comprises of 0.1 to 100% w/w of the formulation. A second type of illustrative aerosol generator comprises a metered dose inhaler. Metered dose inhalers are pressurized aerosol dispensers, typically containing a suspension or solution formulation of active ingredient in a liquified propellant. During use, these devices discharge the formulation through a valve adapted to deliver a metered volume, typically from 10 to 150 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one of more co-solvents, for example, ethanol, surfactants, such as oleic acid or sorbitan trioleate, antioxidants and suitable flavoring agents.

The aerosol, whether formed from solid or liquid particles, may be produced by the aerosol generator at a rate of from about 10 to 150 liters per minute, more preferable from 30 to 150 liters per minute, and most preferably about 60 liters per minute. Aerosols containing greater amounts of medicament may be administered more rapidly.

The dosage of the active compounds disclosed herein will vary depending on the condition being treated and the state of the subject, but generally may be from about 0.01, 0.03, 0.05, 0.1 to 1, 5, 10 or 20 mg of the pharmaceutic agent, deposited on the airway surfaces. The daily dose may be divided among one or multiple unit dose administrations. The goal is to achieve a concentration of the pharmaceutic agents on lung airway surfaces of between 10⁻⁹-10⁴ M.

In another embodiment, they are administered by administering an aerosol suspension of respirable or non-respirable particles (preferably non-respirable particles) comprised of active compound, which the subject inhales through the nose. The respirable or non-respirable particles may be liquid or solid. The quantity of active agent included may be an amount of sufficient to achieve dissolved concentrations of active agent on the airway surfaces of the subject of from about 10⁻⁹, 10⁻⁸, or 10⁻⁷ to about 10⁻³, 10⁻¹ moles/liter, and more preferably from about 10⁻⁹ to about 10⁻⁴ moles/liter.

The dosage of active compound will vary depending on the condition being treated and the state of the subject, but generally may be an amount sufficient to achieve dissolved concentrations of active compound on the nasal airway surfaces of the subject from about 10⁻⁹, 10⁻⁸, 10⁻⁷ to about 10⁻³, 10⁻², or 10⁻¹ moles/liter, and more preferably from about 10⁻⁷ to about 10⁻⁴ moles/liter. Depending upon the solubility of the particular formulation of active compound administered, the daily dose may be divided among one or several unit dose administrations. The daily dose by weight may range from about 0.01, 0.03, 0.1, 0.5 or 1.0 to 10 or 20 milligrams of active agent particles for a human subject, depending upon the age and condition of the subject. A currently preferred unit dose is about 0.5 milligrams of active agent given at a regimen of 2-10 administrations per day, The dosage may be provided as a prepackaged unit by any suitable means (e.g., encapsulating a gelatin capsule).

In one embodiment of the invention, the particulate active agent composition may contain both a free base of active agent and a pharmaceutically acceptable salt to provide both early release and sustained release of active agent for dissolution into the mucus secretions of the nose. Such a composition serves to provide both early relief to the patient, and sustained relief over time. Sustained relief, by decreasing the number of daily administrations required, is expected to increase patient compliance with the course of active agent treatments.

Pharmaceutical formulations suitable for airway administration include formulations of solutions, emulsions, suspensions and extracts. See generally, J. Nairn, Solutions, Emulsions, Suspensions and Extracts, in Remington: The Science and Practice of Pharmacy, chap. 86 (19^{th} ed. 1995), incorporated herein by reference. Pharmaceutical formulations suitable for nasal administration may be prepared as described in U.S. Pat. No. 4,389,393 to Schor; U.S. Pat. No. 5,707,644 to Illum; U.S. Pat. No. 4,294,829 to Suzuki; and U.S. Pat. No. 4,835,142 to Suzuki, the disclosures of which are incorporated by reference herein in their entirety.

Mists or aerosols of liquid particles comprising the active compound may be produced by any suitable means, such as by a simple nasal spray with the active agent in an aqueous pharmaceutically acceptable carrier, such as a sterile saline solution or sterile water. Administration may be with a pressure-driven aerosol nebulizer or an ultrasonic nebulizer. See e.g. U.S. Pat. Nos. 4,501,729 and 5,656,256, both of which are incorporated herein by reference. Suitable formulations for use in a nasal droplet or spray bottle or in nebulizers consist of the active ingredient in a liquid carrier, the active ingredient comprising up to 40% w/w of the formulation, but preferably less than 20% w/w. Typically the carrier is water (and most preferably sterile, pyrogen-free water) or dilute aqueous alcoholic solution, preferably made in a 0.12% to 0.8% solution of sodium chloride. Optional additives include preservatives if the formulation is not made sterile, for example, methyl hydroxybenzoate, antioxidants, flavoring agents, volatile oils, buffering agents, osmotically active agents (e.g. mannitol, xylitol, erythritol) and surfactants.

Compositions containing respirable or non-respirable dry particles of micronized active agent may be prepared by grinding the dry active agent with a mortar and pestle, and then passing the micronized composition through a 400 mesh screen to break up or separate out large agglomerates.

The particulate composition may optionally contain a dispersant which serves to facilitate the formation of an aerosol. A suitable dispersant is lactose, which may be blended with the active agent in any suitable ratio (e.g., a 1 to 1 ratio by weight).

The compounds of formula (I) may be synthesized according to procedures known in the art. A representative synthetic procedure is shown in the scheme below:

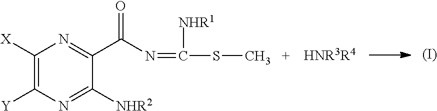

These procedures are described in, for example, E. J. Cragoe, “The Synthesis of Amiloride and its Analogs” (Chapter 3) in Amiloride and Its Analogs, pp. 25-36, incorporated herein by reference. Other methods of preparing the compounds are described in, for example, U.S. Pat. No. 3,313,813, incorporated herein by reference, See in particular Methods A, B, C, and D described in U,S. Pat. No. 3,313,813. Other methods useful for the preparation of these compounds, especially for the preparation of the novel HNR³R⁴ fragment are described in, for example, U.S. Pat. No. 6,858,614, U.S. Pat. No. 6,858,615, and U.S. Pat. No. 6,903,105, incorporated herein by reference.

Several assays may be used to characterize the compounds of the present invention. Representative assays are described below.

1. In Vitro Measure of Epithelial Sodium Channel Block and Beta Agonist Activity

To assess the potency of epithelial sodium channel block and beta agonist activity each compound was tested using two separate experimental procedures with similar methodology.

To assess epithelial sodium channel blocker potency the compounds of the present invention involves the determination of lumenal drug inhibition of airway epithelial sodium currents measured under short circuit current (I_{SC}) using airway epithelial monolayers mounted in Ussing chambers. Cells obtained from freshly excised human, or dog airways are seeded onto porous 0.4 micrometer Transwell® Permeable Supports (Corning Inc. Acton, Mass.), cultured at air-liquid interface (ALI) conditions in hormonally defined media, and assayed for sodium transport activity (I_{SC}) while bathed in Krebs Bicarbonate Ringer (KBR) in Ussing chambers. All test drug additions are to the lumenal bath with approximately half-log dose additions (from 1×10⁻¹¹ M to 6×10⁻⁵ M), and the cumulative change in I_{SC} (decreases) recorded. All drugs are prepared in dimethyl sulfoxide as stock solutions at a concentration of approximately 1×10⁻² and stored at −20° C. Six preparations are typically run in parallel; one preparation per run incorporates 552-02 as a positive control. Before the start of the concentration-effect relationship propranolol, a non-selective beta agonist blocker, was applied to the lumenal bath (10 μM) to inhibit the beta agonist component of the designer multiple ligand (DML). All data from the voltage clamps are collected via a computer interface and analyzed off-line.

Concentration-effect relationships for all compounds are considered and analyzed Using GraphPad Prism version 100 for Windows, GraphPad Software, San Diego Calif. USA. IC₅₀ values, maximal effective concentrations, are calculated and compared to the 552-02 potency as a positive control.

To assess beta agonist activity the compounds of the present invention involves the determination of lamellal drug addition to promote airway epithelial anion currents measured under short circuit current (I_{SC}) using airway epithelial monolayers mounted in Ussing chambers, Cells obtained from freshly excised human, dog, or sheep airways are seeded onto porous 0.4 micron Transwell® Permeable Supports (Corning), cultured at air-liquid interface (ALI) conditions in hormonally defined media, and assayed for anion secretion (I_{SC}) while bathed in Krebs Bicarbonate Ringer (KBR) in Ussing chambers. All test drug additions are to the lumenalth ba with approximately half-log dose additions (from 8×10⁻¹⁰ M to 6.5×10⁻⁵ M), and the cumulative change in I_{SC} (excitation) recorded. All drugs are prepared in dimethyl sulfoxide as stock solutions at a concentration from 1×10⁻¹ to 1×10⁻² M and stored at −20′ C. Six preparations are typically run in parallel; one preparation per run incorporates either formoterol, salmeterol, or another well recognized beta agonists as a positive control depending on the anolog incorporated in the compound being tested. Before the start of the concentration-effect relationship 552-02 a potent sodium channel blocker was applied to the apical surface (1 μM) to eliminate changes in Isc caused by sodium absorption, All data from the voltage clamps are collected via a computer interface and analyzed off-line.

Concentration-effect relationships for all compounds are considered and analyzed Using GraphPad Prism version 3.00 for Windows, GraphPad Software, San Diego Calif. USA. EC₅₀ values, maximal effective concentrations, are calculated and compared to either formoterol or salbutamol as the positive control.

2, In Vitro Assay of Compound Absorption n and Biotransformation by Airway Epithelia

Airway epithelial cells have the capacity to metabolize drugs during the process of transepithelial absorption. Further, although less likely, it is possible that drugs can be metabolized on airway epithelial surfaces by specific ectoenzyme activities. Perhaps more likely as an ecto-surface event, compounds may be metabolized by the infected secretions that occupy the airway lumens of patients with lung disease, e.g. cystic fibrosis. Thus, a series of assays are performed to characterize any compound biotransformation (metabolism or conjugation) that results from the interaction of test compounds with human airway epithelia and/or human airway epithelial lumenal products.

In the first series of assays, the interaction of test compounds in KBR as an “ASL” stimulant are applied to the apical surface of human airway epithelial cells grown in the Transwell® Permeable Supports (Corning), insert system. For most compounds, metabolism or conjugation (generation of new species is tested for using high performance liquid chromatography (HPLC) to resolve chemical species and the endogenous fluorescence properties of these compounds to estimate the relative quantities of test compound and novel metabolites. For a typical assay, a test solution (1 mL KBR, containing 100 μM test compound) is placed on the epithelial lumenal surface. Sequential 5 to 600 μl samples are obtained from the lumenal and serosal compartments respectively for HPLC analysis of (1) the mass of testcompound permeating from the lumenal to serosal bath and (2) the potential formation of metabolites from the parent compound. From the HPLC data, the rate of and/or formation of novel metabolite compounds on the lumenal surface and the appearance of test compound and/or novel metabolite in the basolateral solution is quantitated based on internal standards. The data relating the chromatographic mobility of potential novel metabolites with reference to the parent compound are also quantitated.

To analyze the potential metabolism of test compounds by CF sputum, a “representative” mixture of expectorated CF sputum obtained from 10 CF patients (under IRB approval) has been collected. The sputum has been be solubilized in a 1:5 mixture of KBR solution with vigorous vortexing, following which the mixture was split into a “neat” sputum aliquot and an aliquot subjected to ultracentrifugation so that a “supernatant” aliquot was obtained (neat=cellular; supernatant=liquid phase). Typical studies of compound metabolism by CF sputum involve the addition of known masses of test compound to “neat” CF sputum and aliquots of CF sputum “supernatant” incubated at 37° C., followed by sequential sampling of aliquots from each sputum type for characterization of compound stability/metabolism by HPLC analysis as described above. As above, analysis of compound disappearance, rates of formation of novel metabolities, and HPLC mobilities of novel metabolites are then performed.

### Methods

### Pharmacological Effects and Mechanism of Action of the Drug in Animals

The effect of compounds for enhancing mucociliary clearance (MCC) can be measured using an in vivo model described by Sabater et al., Journal of Applied Physiology, 1999, 87(6) pp. 2191-2196, incorporated herein by reference.

Animal Preparation: Adult ewes up to 75 Kg were placed in a restraint and positioned upright using a specialized body harness. The heads of the animals were immobilized, and local anesthesia of the nasal passage was provided (2% lidocaine) prior to nasal intubation (7.5 mm-I.D. endotracheal tube (ETT) (Mallinekrodt Medical, St. Louis, Mo.). The cuff of the ETT was placed just below the vocal cords. After intubation, the animals were allowed to equilibrate for approximately 20 min before MCC measurements began.

Sheep MCC in vivo Measurement: Aerosols of sulfur colloid raliolabled with technetium (^{99m}Tc-SC 3.1 mg/mL, ˜10-15 mCi) were generated by a Raindrop Nebulizer (Nellcor Puritan Bennett, Pleasanton, Calif.) which produces a median aerodynamic droplet diameter of 3.6 μm. The nebulizer was connected to a dosimeter system consisting of a solenoid valve and a source of compressed air (20 psi). The output of the nebulizer was directed into a T piece, with one end attached to a respirator (Harvard apparatus, South Natick, Mass.). The system was activated for 1 second at the onset of the respirator's inspiratory cycle. The tidal volume was set at 300 mL, with an inspiratory-to-expiratory ratio of 1:1, and a rate of 20 breaths/min, to maximize central airway deposition. The sheep breathed the ^{9m}Tc-SC aerosol for up to 5 min. Following tracer deposition, a gamma camera was used to measure the clearance of ^{99m}Tc-SC from the airways. The camera was positioned above the animal's back with the sheep in its natural upright position in the harness. The field of the image was perpendicular to the animal's spinal cord. External radiolabled markers were placed on the sheep to facilitate proper alignment of the gamma camera. A region of interest was traced over the image corresponding to the right lung of the sheep and counts were recorded. The counts were corrected for decay and expressed as a percentage of radioactivity present in the baseline image. The left lung was excluded from the analysis because the outline of the lung was superimposed over the stomach and counts could be affected by swallowed ^{90m}Tc-SC-labeled mucus. All deposition images were stored on a computer interfaced to the gamma camera. The protocol included a baseline deposition image obtained immediately post radio-aerosol administration. After acquisition of baseline images, either 4 mL of H₂O (vehicle), formoterol (3 mM), or novel chemical entity (3 mM) were aerosolized using the Pari LC JetPlus nebulizer to free-breathing sheep using two separate protocols. Protocol 1, acquired data immediately after dosing (time 0 to 1 hour), and indicated the immediate physiological response ‘short-term efficacy’ protocol 2, acquired data 4 hours post dosing indicated compound durability and ‘long-term efficacy’. The nebulizer had a flow rate of 8 L/min and the time to deliver the solution was 10-12 min. On the completion of compound administration, the animal was immediately extubated to prevent false elevations in counts due to aspiration of excess ^{99m}Tc-SC-labeled mucus from the ETT. Serial measurements of ^{99m}Tc-SC retained in the lung were obtained over a 1 hour period at 5 min intervals. A washout period of at least 7 days (half life of ^{99m}Tc=6 h) separated studies with the different agents.

Statistical Analysis: Data from the in vivo sheep MCC assays were analyzed using a two way ANOVA with repeated measures, followed by slope analysis of the linear regression of the retention vs time plot using an ANOCOVA to compare slopes, and if needed a multiple comparison test (Newman-Keuis). The percent activity retained (post 4 hours) was calculated by dividing the slope value from protocol 2 by the slope value obtained in protocol 1 and multiplying by 100%.

Animal Preparation: Adult ewes (ranging in weight from 25 to 35 kg) were restrained in an upright position in a specialized body harness adapted to a modified shopping cart. The animals' heads were immobilized and local anesthesia of the nasal passage was induced with 2% lidocaine. The animals were then nasally intubated with a 7.5 mm internal diameter endotracheal tube (ETT). The cuff of the ETT was placed just below the vocal cords and its position was verified with a flexible bronchoscope. After intubation the animals were allowed to equilibrate for approximately 20 minutes prior to initiating measurements of mucociliary clearance.

Administration of Radio-aerosol: Aerosols of ^{99m}Tc-Human serum albumin (3.1 mg/ml; containing approximately 20 mCi) were generated using a Raindrop Nebulizer which produces a droplet with a median aerodynamic diameter of 3.6 μm. The nebulizer was connected to a dosimetry system consisting of a solenoid valve and a source of compressed air (20 psi). The output of the nebulizer was directed into a plastic T connector; one end of which was connected to the endotracheal tube, the other was connected to a piston respirator. The system was activated for one second at the onset of the respirator's inspiratory cycle. The respirator was set at a tidal volume of 500 mL, an inspiratory to expiratory ratio of 1:1, and at a rate of 20 breaths per minute to maximize the central airway deposition. The sheep breathed the radio-labeled aerosol for 5 minutes. A gamma camera was used to measure the clearance of ^{99m}Tc-Human serum albumin from the airways. The camera was positioned above the animal=s back with the sheep in a natural upright position supported in a cart so that the field of image was perpendicular to the animal=s spinal cord. External radio-labeled markers were placed on the sheep to ensure proper alignment under the gamma camera. All images were stored in a computer integrated with the gamma camera. A region of interest was traced over the image corresponding to the right lung of the sheep and the counts were recorded. The counts were corrected for decay and expressed as percentage of radioactivity present in the initial baseline image. The left lung was excluded from the analysis because its outlines are superimposed over the stomach and counts can be swallowed and enter the stomach as radio-labeled mucus.

Treatment Protocol (Assessment of activity at t-zero): A baseline deposition image was obtained immediately after radio-aerosol administration. At time zero, after acquisition of the baseline image, vehicle control (distilled water), positive control (amiloride), or experimental compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were extubated immediately following delivery of the total dose in order to prevent false elevations in counts caused by aspiration of excess radio-tracer from the ETT. Serial images of thc lung were obtained at 15-minute intervals during the first 2 hours after dosing and hourly for the next 6 hours after dosing for a total observation period of 8 hours. A washout period of at least 7 days separated dosing sessions with different experimental agents.

Treatment Protocol (Assessment of Activity at t-4 hours): The following variation of the standard protocol was used to assess the durability of response following a single exposure to vehicle control (distilled water), positive control compounds (amiloride or benzamil), or investigational agents. At time zero, vehicle control (distilled water), positive control (amiloride), or investigational compounds were aerosolized from a 4 ml volume using a Pari LC JetPlus nebulizer to free-breathing animals. The nebulizer was driven by compressed air with a flow of 8 liters per minute, The time to deliver the solution was 10 to 12 minutes, Animals were restrained in an upri position in a specialized body harness for 4 hours. At the end of the 4-hour period animals received a single dose of aerosolized ^{99m}Tc-Human serum albumin (3.1 mg/ml; containing approximately 20 mCi) from a Raindrop Nebulizer, Animals were extubated immediately following delivery of the total dose of radio-tracer. A baseline deposition image was obtained immediately after radio-aerosol administration. Serial images of the lung were obtained at 15-minute intervals during the first 2 hours after administration of the radio-tracer (representing hours 4 through 6 after drug administration) and hourly for the next 2 hours after dosing for a total observation period of 4 hours. A washout period of at least 7 days separated dosing sessions with different experimental agents.

Statistics: Data were analyzed using SYSTAT for Windows, version 5. Data were analyzed using a two-way repeated ANOVA (to assess overall effects), followed by a pared t-test to identify differences between specific pairs. Significance was accepted when P was less than or equal to 0.05. Slope values (calculated from data collected during the initial 45 minutes after dosing in the t-zero assessment) for mean MCC curves were calculated using linear least square regression to assess differences in the initial rates during the rapid clearance phase.

Obviously, numerous modifications and variations of the present invention are possible in light of the above teachings. It is therefore to be understood that within the scope of the appended claims, the invention may be practiced otherwise than as specifically described herein.

1-97. (canceled)

98. A compound represented by formula (I): 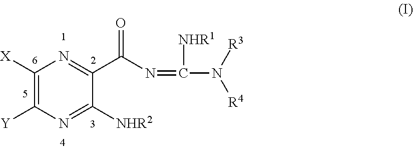
wherein
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰,
—(CH₂)ₙ(CHOR⁸)(CHOR⁸)ₙ—CH₂OR⁸, —(CH₂CH₂O)_{m—R}⁸,
—(CH₂CH₂O)ₘ—CH₂CH₂NR⁷R¹⁰, —(CH₂)ₙ—C(═O)NR⁷R¹⁰, —(CH₂)ₙ—Z_{g}—R⁷, —(CH₂)ₘ—NR¹⁰—CH₂(CHOR⁸)(CHOR⁸)ₙ—CH₂OR⁸, —(CH₂)ₙ—CO₂R⁷, or 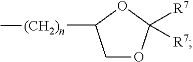
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 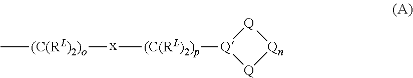
wherein
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —-glucuronide, —O-glucose, 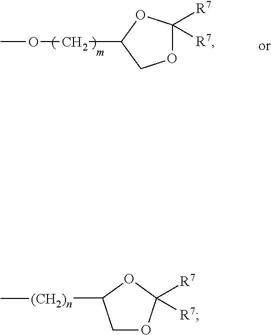
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰),
CHNR⁷R¹⁰, or represents a single bond;
wherein each R⁵ is, independently,
Link —(CH₂)ₙ—CR¹¹R¹¹—CAP, Link —(CH₂)ₙ(CHOR⁸)(CHOR⁸)ₙ—CR¹¹R¹¹—CAP, Link —(CH₂CH₂O)ₘ—CH₂—CR¹¹R¹¹—CAP, Link —(CH₂CH₂O)ₘ—CH₂CH₂—CR¹¹R¹¹—CAP, Link −(CH₂)ₙ—(Z)_{g}—CR¹¹R¹¹—CAP, Link —(CH₂)ₙ(Z)_{g}—(CH₂)ₘ—CR¹¹R¹¹—CAP, Link −(CH₂)ₙ—NR¹³—CH₂(CHOR⁸)(CHOR⁸)ₙ—CR¹¹R¹¹—CAP, Link −(CH₂)ₙ—(CHOR⁸)ₘCH₂—NR¹³—(Z)_{g}—CR¹¹R¹¹—CAP, Link —(CH₂)ₙNR¹³—(CH₂)ₘ(CHOR⁸)ₙCH₂NR¹³—(Z)_{g}—CR¹¹R¹¹—CAP, Link —(CH₂)ₘ—(Z)_{g}—(CH₂)ₘ—CR¹¹R¹¹—CAP, Link NH—C(═O)—NH—(CH₂)ₘ—CR¹¹R¹¹—CAP, Link —(CH₂)ₘ—C(═O)NR¹³—(CH₂)ₘ—CR¹¹R¹¹—CAP, Link —(CH₂)ₙ—(Z)_{g}—(CH₂)ₘ—(Z)_{g}—CR¹¹R¹¹—CAP, Link Z_{g}—(CH₂)ₘ-Het-(CH₂)ₘ—CR¹¹R¹¹—CAP;
each Link is, independently,
—O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ—Z_{g}—(CH₂)ₙ, —S—, —SO—, —SO₂—, SO₂NR⁷—, SO₂NR¹⁰—, -Het-.
each CAP is, independently, 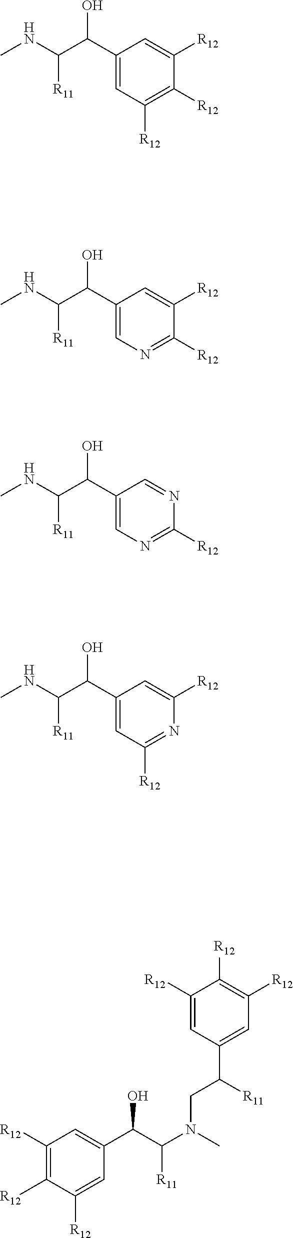
each R⁶ is, independently, —R⁷, —OR⁷, —OR¹¹, —N(R⁷)₂, —(CH₂)ₘ, —OR⁸, —O—(CH₂)ₘ—OR⁸, —(CH₂)_{n——NR}⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ—(Z)_{g}—R⁷, —O—(CH₂)ₘ—(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O -glucuronide, —O-glucose, 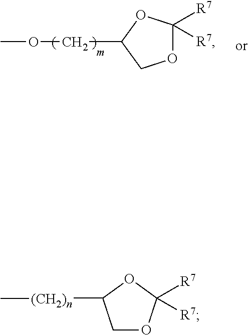
wherein when two R⁶ are —OR¹¹ and are located adjacent to each other on a phenyl ring, the alkyl moieties of the two R⁶ may be bonded together to form a methylenedioxy group;
with the proviso that when at least two —CH₂OR⁸ are located adjacent to each other, the R⁸ groups may be joined to form a cyclic mono- or di-substituted 1,3-dioxane or 1,3-dioxolane,
each R⁷ is, independently, hydrogen, lower alkyl, phenyl, or substituted phenyl;
each R⁸ is, independently, hydrogen, lower alkyl, —C(═O)—R¹¹, glucuronide, 2-tetrahydropyranyl, or 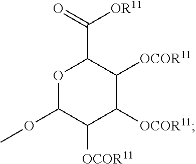
each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R⁷, —C(═O)NR⁷R⁹,
—C(═O)R⁷, or (CH₂)ₘ—(CHOH)ₙ—CH₂OH;
each Z is, independently, CHOH, C(═O), —(CH₂)ₙ—, CHNR¹³R¹³, C═NR¹³, or NR¹³;
each R¹¹ is, independently, hydrogen, lower alkyl, phenyl lower alkyl or substituted phenyl lower alkyl;
each R¹² is independently, —(CH₂)ₙ—SO₂CH₃, —(CH₂)ₙ—CO₂R¹³, —(CH₂)ₙ—C(═O)NR¹³R¹³, —(CH₂)ₙ—C(═O)R¹³, —(CH₂)ₙ—(CHOH)ₙ—CH₂OH, —NH—(CH₂)ₙ—SO₂CH₃ , NH—(CH₂)ₙ—C(═O)R¹¹, NH—C(═O)—NH—C(═O)R¹¹, —C(═O)NR¹³R¹³, —OR¹¹, —NH—(CH₂)ₙ—R¹⁰, —Br, —Cl, —F, —I, SO₂NHR¹¹, —NHR¹³, —NH—C(═O)—NR¹³R¹³, NH—(CH₂)ₙ—SO₂CH₃, NH—(CH₂)ₙ—C(═O)R¹¹, —NH—C(═O)—NH—C(═O)R¹¹, —C(═O)NR¹³R¹³, —OR¹¹, —(CH₂)ₙ—NHR¹³, —NH—C(═O)—NR¹³R¹³, or —NH—(CH₂)ₙ—C(═O)—R¹³;
each R¹³ is, independently, hydrogen, lower alkyl, phenyl, substituted phenyl, 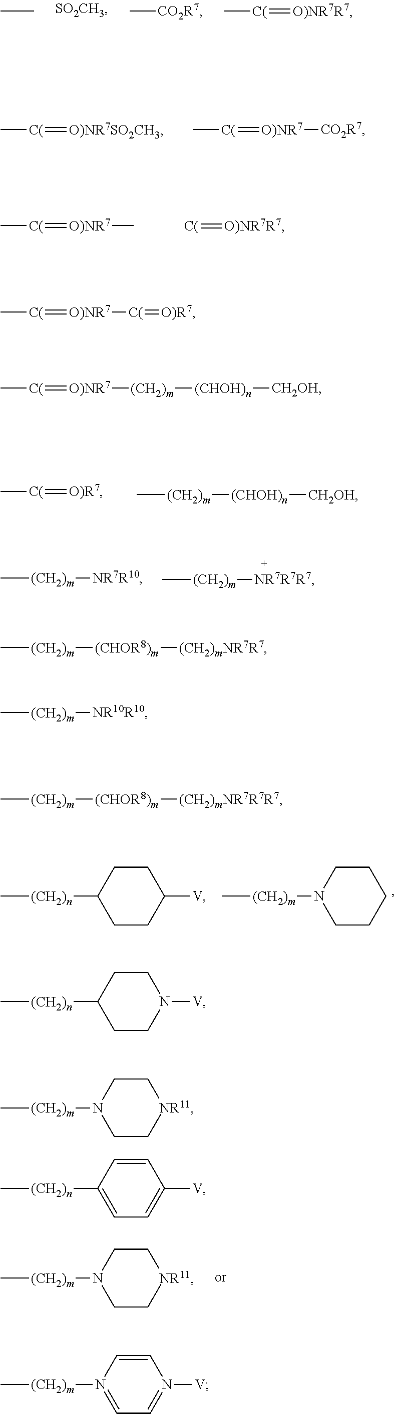
with the proviso that NR¹³R¹³ can be joined on itself to form a group represented by one of the following: 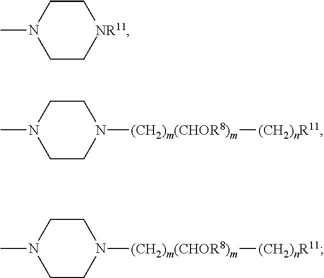
each Het is independently, —NR¹³, —S—, —SO—, —SO₂—, —O—, —SO₂NR¹³—, —NHSO₂—, —NR¹³CO—, or —CONR¹³—;
each g is, independently, an integer from 1 to 6;
each m is, independently, an integer from 1 to 7;
each n is, independently, an integer from 0 to 7;
each Q′ is, independently, CR⁶ or N;
each Q is independently, —C(R⁶R⁵)—, —C(R⁶R⁶)—, —N(R¹⁰)—, —N(R⁷)—, —N(R⁵)—, —S—, —SO—, or —SO₂—,
with the proviso that at least one Q is —C(R⁵R⁶)— or —N(R⁵)—,
with the proviso that at most three Q in a ring is —N(R⁷)—, —N(R⁵)—, —S—, —SO—, or —SO₂—;
each V is, independently, 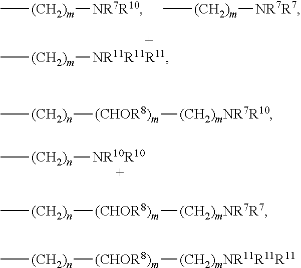
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane;
or a pharmaceutically acceptable salt thereof, and
inclusive of all racemates, enantiomers, diastereomers, tautomers, polymorphs and pseudopolymorphs thereof.

99. The compound of claim 98 wherein Y is —NH₂; each R¹, R², R³, R^{L} and R⁶ is hydrogen; X is chlorine; the sum of o and p is 3 to 6; x represents a bond and at most two Q are a —N(R⁷)—.

100. The compound of claim 98 wherein Y is —NH₂; each R¹, R², R³, R^{L} and R⁶ is hydrogen; X is chlorine; o is 4; p is 0; x represents a bond and at most two Q are a —N(R⁷).

101. The compound of claim 98 wherein each R^{L} is a hydrogen atom; x is a single bond; and at most two Q are —N(R⁷)—.

102. The compound of claim 98 wherein n is an integer from 3 to 6.

103. The compound of claim 98 wherein n is 4.

104. The compound of claim 98 wherein at most two Q are —N(R⁷)—.

105. The compound of claim 98 which is

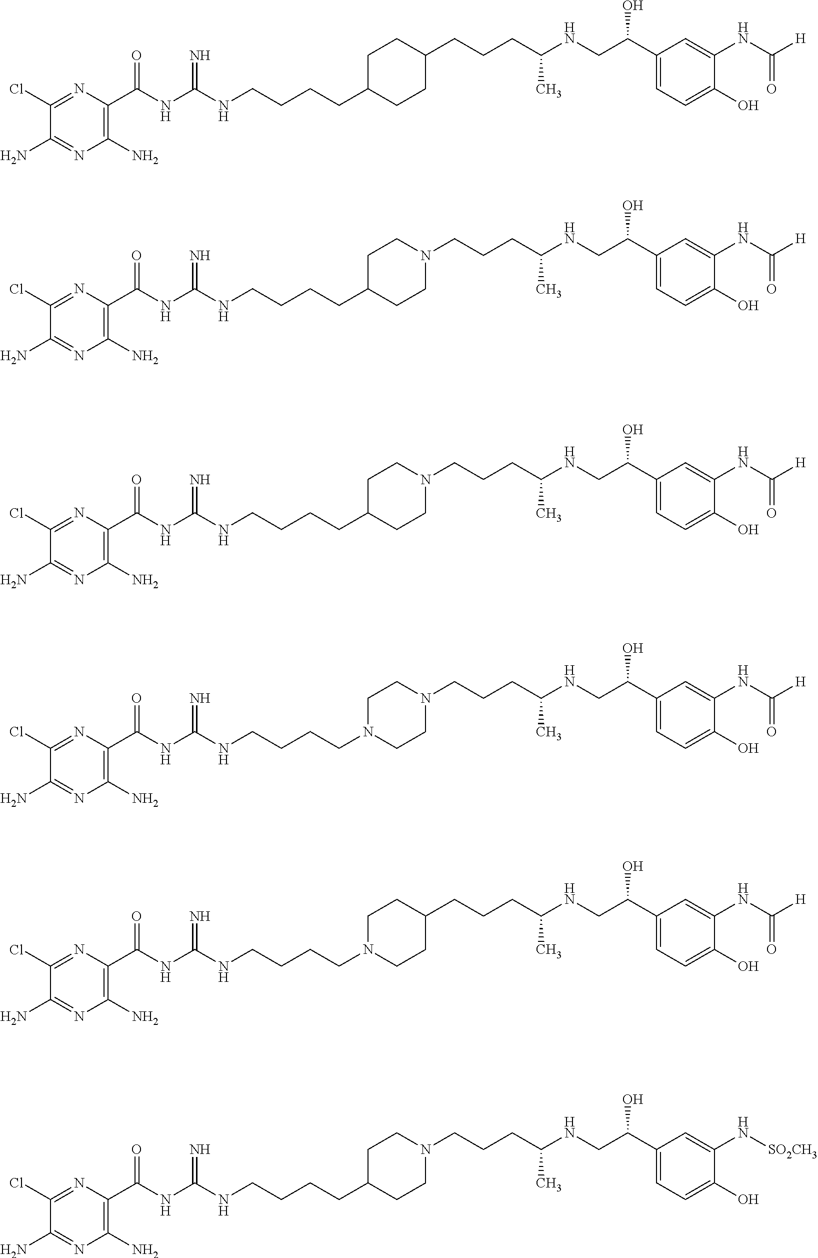

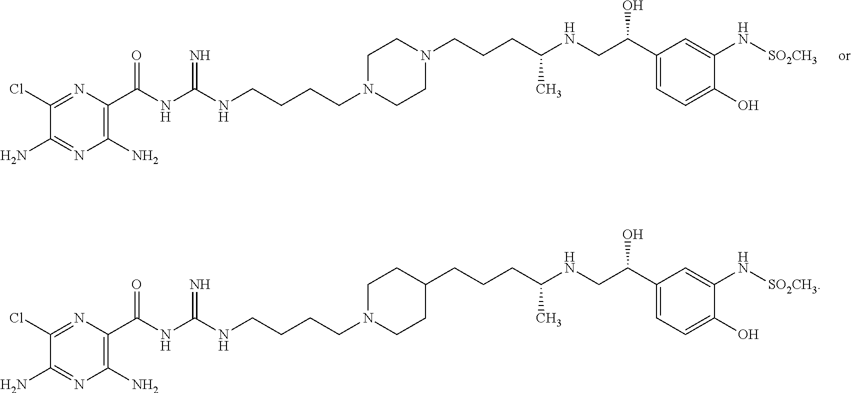

106. A pharmaceutical composition comprising the compound of claim 98 and a pharmaceutically acceptable carrier.

107. A composition according to claim 106 further comprising at least one other agent selected from the group consisting of a P2Y2 agonist, a bronchodilator, a cholinergic bronchodilator, an ionic osmolyte, sodium chloride, an organic osmolyte, mannitol, and an adenosine agonist.

108. A method of blocking sodium channels and activating beta receptors simultaneously comprising contacting sodium channels and beta receptors with an effective amount of the compound of claim 98.

108. A method of treating one or more conditions selected from the group consisting of chronic bronchitis, cystic fibrosis, sinusitis, vaginal dryness, dry eye, Sjogren's disease, distal intestinal obstruction syndrome, dry skin, esophagitis, dry mouth (xerostomia), nasal dehydration, ventilator-induced pneumonia, asthma, primary ciliary dyskinesia, otitis media, chronic obstructive pulmonary disease, emphysema, pneumonia, constipation, chronic diverticulitis, and rhinosinusitis; comprising administering an effective amount of a compound of claim 98 to a human in need thereof.

109. The method of claim 108 wherein the condition is cystic fibrosis.

110. The method of claim 109 wherein the compound is administered as an inhalable aerosol.

111. A method of treating one or more conditions selected from the group consisting of chronic bronchitis, cystic fibrosis, ventilator-induced pneumonia, asthma, chronic obstructive pulmonary disease, emphysema, and pneumonia comprising administering an effective amount of a compound of claim 98 to a human in need thereof.

112. A method of treating asthma or chronic obstructive pulmonary disease, comprising administering an effective amount of a compound of claim 98 to a human in need thereof.

113. The method of claim 124 wherein the compound is administered as an inhalable aerosol.
